(19) United States
(12) Patent Application Publication
(10) Pub. No.: US 2018/0294060 A1
(43) Pub. Date: Oct. 11, 2018

(54) TECHNOLOGICAL DEVICES AND SYSTEMS AND METHODS TO USE THE SAME TO OBTAIN BIOLOGICAL INFORMATION

(71) Applicant: Ghassan S. Kassab, La Jolla, CA (US)

(72) Inventor: Ghassan S. Kassab, La Jolla, CA (US)

(21) Appl. No.: 15/949,568

(22) Filed: Apr. 10, 2018

Related U.S. Application Data
(60) Provisional application No. 62/483,506, filed on Apr. 10, 2017.
(60) Provisional application No. 62/483,508, filed on Apr. 10, 2017.
(60) Provisional application No. 62/483,509, filed on Apr. 10, 2017.
(60) Provisional application No. 62/483,512, filed on Apr. 10, 2017.

(51) Int. Cl. G16H 50/50; G16H 10/65; G16H 50/30

(52) U.S. Cl. CPC G16H 50/50; G16H 50/30; G16H 10/65

(57) ABSTRACT

Technological devices and systems and methods to use the same to obtain biological information. A method referenced herein can be performed, for example, using a processor in communication with a first storage device to execute instructions stored thereon, the method including the steps of accessing a combined simulation stored on the first storage device or a second storage device, the combined simulation comprising a library comprising a plurality of computational simulations; obtaining a first patient-specific parameter and a second patient-specific parameter from a patient; and analyzing the first patient-specific parameter and the second patient-specific parameter in view of the combined simulation to generate a patient-specific outcome comprising a first outcome element and a second outcome element.

### PRIORITY

The present application is related to, and claims the priority benefit of, a) U.S. Provisional Patent Application Ser. No. 62/483,506, filed Apr. 10, 2017, b) U.S. Provisional Patent Application Ser. No. 62/483,508, filed Apr. 10, 2017, c) U.S. Provisional Patent Application Ser. No. 62/483,509, filed Apr. 10, 2017, and d) U.S. Provisional Patent Application Ser. No. 62/483,512, filed Apr. 10, 2017. The contents of each of the aforementioned patent applications are incorporated herein directly and by reference in their entirety.

### BACKGROUND

Patient-specific simulations are complex and not real-time. There is substantial interest in precision medicine where surgical or interventional procedures are tailored to the anatomy and physiology of an individual patient. Patient-specific data are used as input into computational simulations that account for specific imaging information of the patient such as CT, MRI, PET, ultrasound, etc., as well as physiologic measurements such as pressure or flow. The difficulty is that patient-specific simulations to design or virtually plan an optimal procedure cannot be made in real-time given the substantial computational expense of the models. Hence, there is a need for a practical approach to have virtual planning that is real-time to aid in clinical decision making to optimize outcome and prognosis.

Individuals are at risk for various diseases and management of risk factors (RFs) is crucial to good health.

Various studies (e.g., the Framingham study for cardiovascular system) established numerous risk factors for various diseases (heart disease, cancer, stroke, pulmonary embolism, etc.). The clinical and scientific studies have established the probability of various diseases based on the risk factors. Precision medicine requires tailoring of these risk factors to an individual. As with above, knowledge of patient specific data (imaging, physiological parameters, proteomics, genomics, etc.) will enable a greater precisions in disease predilection and ultimately prediction.

Stroke is a huge problem in the United States, with nearly 1 million cases per year. The after-effects of stroke patients can range from mild to severe, should the patient survive the stroke itself. Carotid stenosis can lead to stroke, and early detection and management are crucial.

Individuals are at risk for various diseases and management of risk factors (RFs) is crucial to good health. Various studies (e.g., the Framingham study for cardiovascular system) established numerous risk factors for various diseases (heart disease, cancer, stroke, pulmonary embolism, etc.). The clinical and scientific studies have established the probability of various diseases based on the risk factors. Early indications of disease may enable medical and life style changes that alter the progression of the disease.

Blood pressure (systolic/diastolic) is a direct biomarker of hypertension/hypotension and cardiovascular disease. More frequent assessment of blood pressure can reflect changes in cardiovascular health. The current methodology to assess blood pressure require either a clinical visit or pharmacy (CVS, etc.) to use a pressure cuff. A device to allow an individual to readily obtain blood pressure data would be well received in the marketplace.

### BRIEF SUMMARY

The present disclosure includes disclosure of a method, such as, for example, performed using a processor in communication with a first storage device to execute instructions stored thereon, the method comprising the steps of accessing a combined simulation stored on the first storage device or a second storage device, the combined simulation comprising a library comprising a plurality of computational simulations; obtaining a first patient-specific parameter and a second patient-specific parameter from a patient; and analyzing the first patient-specific parameter and the second patient-specific parameter in view of the combined simulation to generate a patient-specific outcome comprising a first outcome element and a second outcome element.

The present disclosure includes disclosure of a method, further comprising the step of performing a medical procedure upon the patient based upon the patient-specific outcome.

The present disclosure includes disclosure of a method, wherein the plurality of computational simulations is generated using generic information and model patient information.

The present disclosure includes disclosure of a method, wherein the generic information comprises one or more of field equations, and wherein the model patient information comprises a patient-specific geometry or structure of an organ or a vessel.

The present disclosure includes disclosure of a method, wherein at least one computational simulation of the plurality of computational simulation is selected from the group consisting of a cardiovascular simulation, a cardiac simulation, a blood flow simulation, a stent simulation, a stent graft simulation, and a cardiac mechanics simulation.

The present disclosure includes disclosure of a method, wherein a computational simulation of the plurality of computational simulations comprises a first aspect relating to patient age and a second aspect relating to patient weight.

The present disclosure includes disclosure of a system, comprising a processor in communication with a storage device and configured to execute instructions stored thereon; and a combined simulation stored on the storage device or a second storage device, the combined simulation comprising a library comprising a plurality of computational simulations; wherein the system is configured to obtain a first patient-specific parameter and a second patient-specific parameter from a patient; and wherein the processor is configured to analyze the first patient-specific parameter and the second patient-specific parameter in view of the combined simulation to generate a patient-specific outcome comprising a first outcome element and a second outcome element.

The present disclosure includes disclosure of a method, such as, for example, performed using a processor in communication with a first storage device to execute instructions stored thereon, the method comprising the steps of accessing an electronic medical record on the first storage device or a second storage device, the electronic medical record comprising a first patient-specific parameter, a second patient-specific parameter, and a third patient-specific parameter; generating a risk factor baseline based upon the electronic medical record and reporting a risk factor should a risk factor be identified upon generation of the risk factor baseline; operating a portable device to receive patient data from a remote device comprising one or more sensors or electrodes positioned upon or within a patient; incorporating the patient data into the electronic medical record to form an updated electronic medical record; and generating a risk factor level based upon the updated electronic medical record and reporting a risk factor change or a new risk factor should the risk factor change or the new risk factor be identified upon generation of the risk factor level.

The present disclosure includes disclosure of a method, wherein the first patient-specific parameter is selected from the group consisting of a heart rate, a blood pressure, a blood glucose level, and a cholesterol level.

The present disclosure includes disclosure of a method, wherein the first patient-specific parameter is historical or current.

The present disclosure includes disclosure of a method, wherein the portable device is selected from the group consisting of a smartphone and a smartwatch.

The present disclosure includes disclosure of a method, further comprising the steps of operating the portable device again to receive additional patient data from the remote device; incorporating the additional patient data into the electronic medical record to form a further updated electronic medical record; and generating an additional risk factor level based upon the further updated electronic medical record and reporting an additional risk factor change or an additional new risk factor should the additional risk factor change or the additional new risk factor be identified upon generation of the additional risk factor level.

The present disclosure includes disclosure of a system, comprising a processor in communication with a storage device and configured to execute instructions stored thereon; and an electronic medical record stored on the storage device or a second storage device, the electronic medical record comprising a first patient-specific parameter, a second patient-specific parameter, and a third patient-specific parameter; wherein the system is configured to generate a risk factor baseline based upon the electronic medical record and reporting a risk factor should a risk factor be identified upon generation of the risk factor baseline; wherein the system is configured to receive patient data from a remote device comprising one or more sensors or electrodes positioned upon or within a patient; wherein the system is configured to incorporate the patient data into the electronic medical record to form an updated electronic medical record; and wherein the system is configured to generate a risk factor level based upon the updated electronic medical record and reporting a risk factor change or a new risk factor should the risk factor change or the new risk factor be identified upon generation of the risk factor level.

The present disclosure includes disclosure of a method, such as, for example, performed using a processor in communication with a first storage device to execute instructions stored thereon, the method comprising the steps of operating a first device comprising one or more sensors or electrodes configured to detect physiological information so to obtain physiological information/data from a user; comparing the physiological information/data with library data or with previously-obtained data to generate a comparison result; and alerting the user should the comparison result be at or above a first threshold level.

The present disclosure includes disclosure of a method, wherein the physiological information comprises sound vibrations and/or turbulence created by steno sis or constriction of a vessel lumen, wherein the physiological information/data comprises vibration and/or turbulence data, and wherein the comparison result identifies a carotid stenosis or a degree of carotid stenosis near, at, or above the first threshold level.

The present disclosure includes disclosure of a method, further comprising the step of obtaining medical attention and treatment based upon the identified carotid stenosis or the degree of carotid stenosis near, at, or above the first threshold level.

The present disclosure includes disclosure of a method, wherein the step of comparing is performed to compare the vibration and/or turbulence data with library data comprising a plurality of computational simulations generated using generic information and model patient information.

The present disclosure includes disclosure of a method, wherein the physiological information comprises blood pressure, wherein the physiological information/data comprises blood pressure data/information, and wherein the comparison result identifies a blood pressure measurement near, at, or above the first threshold level or at, near, or below a second, lower threshold level.

The present disclosure includes disclosure of a method, further comprising the step of obtaining medical attention and treatment for hypotension should the comparison result identify a blood pressure measurement at, near, or below the second, lower threshold level.

The present disclosure includes disclosure of a method, further comprising the step of obtaining medical attention and treatment for hypertension or cardiovascular disease should the comparison result identify a blood pressure measurement at, near, or above the first threshold level.

The present disclosure includes disclosure of a method, wherein the step of comparing is performed to compare the blood pressure data/information with library data comprising a plurality of computational simulations generated using generic information and model patient information.

The present disclosure includes disclosure of a method, wherein the first device is configured as a smartwatch, and wherein the step of operating the first device is performed while the first device is worn upon a wrist of the user.

The present disclosure includes disclosure of a method, such as, for example, performed using a processor in communication with a first storage device to execute instructions stored thereon, the method comprising the steps of operating a first device comprising one or more sensors or electrodes configured to detect sound vibrations and/or turbulence created by stenosis or constriction of a vessel lumen to obtain vibration and/or turbulence data from a user; comparing the vibration and/or turbulence data with library data or with previously-obtained data to generate a comparison result; and alerting the user should the comparison result identify a carotid stenosis or a degree of carotid stenosis near, at, or above a threshold stenosis level.

The present disclosure includes disclosure of a method, further comprising the step of obtaining medical attention and treatment based upon the identified carotid stenosis or the degree of carotid stenosis near, at, or above the threshold stenosis level.

The present disclosure includes disclosure of a method, wherein the step of comparing is performed to compare the vibration and/or turbulence data with library data comprising a plurality of computational simulations generated using generic information and model patient information.

The present disclosure includes disclosure of a device, comprising a processor in communication with a storage device and configured to execute instructions stored thereon; and one or more sensors or electrodes configured to detect sound vibrations and/or turbulence created by stenosis or constriction of a vessel lumen to obtain vibration and/or turbulence data from a user; wherein the device is configured to compare the vibration and/or turbulence data with library data or with previously-obtained data to generate a comparison result; and wherein the device is configured to alert the user should the comparison result identify a carotid stenosis or a degree of carotid stenosis near, at, or above a threshold stenosis level.

The present disclosure includes disclosure of a method, such as, for example, performed using a processor in communication with a first storage device to execute instructions stored thereon, the method comprising the steps of operating a first device comprising one or more sensors or electrodes configured to detect blood pressure so to obtain blood pressure data/information from a user; comparing the blood pressure data/information with library data or with previously-obtained data to generate a comparison result; and alerting the user should the comparison result identify a blood pressure measurement above an upper threshold level or below a lower threshold level.

The present disclosure includes disclosure of a method, further comprising the step of obtaining medical attention and treatment for hypotension should the comparison result identify a blood pressure measurement at or below the lower threshold level.

The present disclosure includes disclosure of a method, further comprising the step of obtaining medical attention and treatment for hypertension or cardiovascular disease should the comparison result identify a blood pressure measurement at or above the lower threshold level.

The present disclosure includes disclosure of a method, wherein the step of comparing is performed to compare the blood pressure data/information with library data comprising a plurality of computational simulations generated using generic information and model patient information.

The present disclosure includes disclosure of a method, wherein the first device is configured as a smartwatch, and wherein the step of operating the first device is performed while the first device is worn upon a wrist of the user.

The present disclosure includes disclosure of a device, comprising a processor in communication with a storage device and configured to execute instructions stored thereon; and one or more sensors or electrodes configured to detect blood pressure so to obtain blood pressure data/information; wherein the device is configured to compare the blood pressure data/information with library data or with previously-obtained data to generate a comparison result; and wherein the device is configured to alert the user should the comparison result identify a blood pressure measurement above an upper threshold level or below a lower threshold level.

### BRIEF DESCRIPTION OF THE DRAWINGS

The disclosed embodiments and other features, advantages, and disclosures contained herein, and the matter of attaining them, will become apparent and the present disclosure will be better understood by reference to the following description of various exemplary embodiments of the present disclosure taken in conjunction with the accompanying drawings, wherein:

FIG. 1 is a schematic diagrammatic view of a network system in which embodiments of the present disclosure may be utilized;

FIG. 2 is a block diagram of a computing system (either a server or client, or both, as appropriate), with optional input devices (e.g., keyboard, mouse, touch screen, etc.) and output devices, hardware, network connections, one or more processors, and memory/storage for data and modules, etc. which may be utilized in conjunction with embodiments of the present disclosure;

FIG. 3 shows a block diagram of aspects of a system constructed to be used in connection with patient-specific information inputs, according to an exemplary embodiment of the present disclosure;

FIG. 4 shows a block diagram of individual aspects of a computational simulation, according to an exemplary embodiment of the present disclosure;

FIG. 5 shows a block diagram of individual outcome elements of a patient-specific outcome, according to an exemplary embodiment of the present disclosure;

FIG. 6 shows a block diagram of individual patient-specific parameters forming part of an electronic medical record, according to an exemplary embodiment of the present disclosure;

FIG. 7 shows a block diagram of portions of a system including a first device and a remote device, according to an exemplary embodiment of the present disclosure;

FIG. 8 shows a block diagram of steps of a method to obtain data to determine risk factors in real time, according to an exemplary embodiment of the present disclosure;

FIG. 9 shows a block diagram of portions of a system including a first device, according to an exemplary embodiment of the present disclosure;

FIG. 10 shows a block diagram of aspects of a system constructed to be used in connection with patient-specific information inputs, according to an exemplary embodiment of the present disclosure;

FIG. 11 shows a block diagram of steps of a method, according to an exemplary embodiment of the present disclosure;

FIG. 12 shows a top/front view a first device configured as a smartwatch, according to an exemplary embodiment of the present disclosure;

FIG. 13 shows a back/underside view a first device configured as a smartwatch, according to an exemplary embodiment of the present disclosure;

FIG. 14 shows a back/underside view a first device configured as a smartwatch with automatic tightening, according to an exemplary embodiment of the present disclosure;

FIG. 15 shows a block diagram of aspects of a system constructed to be used in connection with patient-specific information inputs, according to an exemplary embodiment of the present disclosure; and

FIG. 16 shows a block diagram of steps of a method, according to an exemplary embodiment of the present disclosure

An overview of the features, functions and/or configurations of the components depicted in the various figures will now be presented. It should be appreciated that not all of the features of the components of the figures are necessarily described. Some of these non-discussed features, such as various couplers, etc., as well as discussed features are inherent from the figures themselves. Other non-discussed features may be inherent in component geometry and/or configuration.

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of this disclosure is thereby intended.

The present disclosure includes disclosure of systems for use with patient-specific simulations and methods to construct and use the same for patient diagnosis and/or treatment. The present disclosure also includes disclosure of a system that can constantly (in real-time) quantify an individual's risk factors for various diseases based on medical history, exams, biomarkers (anatomical, functional, physiological, molecular, etc.), etc. Such a system can include the use of a smart device, such as a smart phone, in communication with a remote device that can obtain real-time patient data. Furthermore, and as noted above, carotid stenosis can lead to stroke, and early detection and management are crucial. The present disclosure includes disclosure of a system, and use of the same, configured to estimate a degree of carotid stenosis based on sound detection. A solution of the sound equation can provide an estimate of degree of carotid lumen area reduction. In addition, and as noted above, high blood pressure is a direct biomarker for several diseases. The present disclosure includes disclosure of a system, and use of the same, configured to monitor blood pressure over time, such as weekly, daily, or several times a day, such as on a schedule set and/or authorized by the user.

Various techniques and mechanisms of the present disclosure will sometimes describe a connection between two components. Words such as attached, affixed, coupled, connected, and similar terms with their inflectional morphemes are used interchangeably unless the difference is expressly noted or made otherwise clear from the context. These words and expressions do not necessarily signify direct connections, but include connections through mediate components and devices. Indeed, it should be noted that a connection between two components does not necessarily mean a direct, unimpeded connection, as a variety of other components may reside between the two components of note. For example, a workstation may be in communication with a server, but it will be appreciated that a variety of bridges and controllers may reside between the workstation and the server. Consequently, a connection does not necessarily mean a direct, unimpeded connection unless otherwise noted.

Furthermore, wherever feasible and convenient, like reference numerals are used in the figures and the description to refer to the same or like parts or steps. The drawings are in a simplified form and not to precise scale.

The detailed descriptions which follow are presented in part in terms of algorithms and symbolic representations of operations on data bits within a computer memory representing alphanumeric characters or other information. A computer generally includes a processor for executing instructions and memory for storing instructions and data. When a general purpose computer has a series of machine encoded instructions stored in its memory, the computer operating on such encoded instructions may become a specific type of machine, namely a computer particularly configured to perform the operations embodied by the series of instructions. Some of the instructions may be adapted to produce signals that control operation of other machines and thus may operate through those control signals to transform materials far removed from the computer itself. These descriptions and representations are the means used by those skilled in the art of data processing arts to most effectively convey the substance of their work to others skilled in the art.

An algorithm is here, and generally, conceived to be a self consistent sequence of steps leading to a desired result. These steps are those requiring physical manipulations of physical quantities. Usually, though not necessarily, these quantities take the form of electrical or magnetic pulses or signals capable of being stored, transferred, transformed, combined, compared, and otherwise manipulated. It proves convenient at times, principally for reasons of common usage, to refer to these signals as bits, values, symbols, characters, display data, terms, numbers, or the like as a reference to the physical items or manifestations in which such signals are embodied or expressed. It should be kept in mind, however, that all of these and similar terms are to be associated with the appropriate physical quantities and are merely used here as convenient labels applied to these quantities.

Some algorithms may use data structures for both inputting information and producing the desired result. Data structures greatly facilitate data management by data processing systems, and are not accessible except through software systems. Data structures are not the information content of a memory, rather they represent specific electronic structural elements which impart or manifest a physical organization on the information stored in memory. More than mere abstraction, the data structures are specific electrical or magnetic structural elements in memory which simultaneously represent complex data accurately, often data modeling physical characteristics of related items, and provide increased efficiency in computer operation.

Further, the manipulations performed are often referred to in terms, such as comparing or adding, commonly associated with mental operations performed by a human operator. No such capability of a human operator is necessary, or desirable in most cases, in any of the operations described herein which form part of the embodiments of the present application; the operations are machine operations. Indeed, a human operator could not perform many of the machine operations described herein due, at least in part, to the networking and vast distribution capabilities of the present disclosure.

Useful machines for performing the operations of one or more embodiments hereof include general purpose digital computers or other similar devices. In all cases the distinction between the method operations in operating a computer and the method of computation itself should be recognized. One or more embodiments of the present application relate to methods and apparatus for operating a computer in processing electrical or other (e.g., mechanical, chemical) physical signals to generate other desired physical manifestations or signals. The computer and systems described herein operate on software modules, which are collections of signals stored on a media that represents a series of machine instructions that enable the computer processor to perform the machine instructions that implement the algorithmic steps. Such machine instructions may be the actual computer code the processor interprets to implement the instructions, or alternatively may be a higher level coding of the instructions that is interpreted to obtain the actual computer code. The software module may also include a hardware component, wherein some aspects of the algorithm are performed by the circuitry itself rather as a result of an instruction.

Some embodiments of the present disclosure also relate to an apparatus or specific hardware for performing the disclosed operations. This apparatus and/or hardware may be specifically constructed for the required purposes or it may comprise a general purpose computer or related hardware as selectively activated, employed, or reconfigured by a computer program stored in the computer. The algorithms presented herein are not inherently related to any particular computer or other apparatus unless explicitly indicated as requiring particular hardware. In some cases, the computer programs may communicate or relate to other programs or equipment through signals configured to particular protocols which may or may not require specific hardware or programming to interact (e.g., in at least one embodiment, the computer programs use a set of predefined Application Program Interfaces (APIs, defined and described in further detail below)). In particular, various general purpose machines may be used with programs written in accordance with the teachings herein, or it may prove more convenient to construct at least one more specialized apparatus to perform the required method steps. The required structure for a variety of these machines will appear from the description below.

Embodiments of the present invention may deal with “object oriented” software, and particularly with an “object oriented” operating system. The “object oriented” software is organized into “objects,” each comprising a block of computer instructions describing various procedures (“methods”) to be performed in response to “messages” sent to the object or “events” which occur with the object. Such operations include, for example, the manipulation of variables, the activation of an object by an external event, and the transmission of one or more messages to other objects.

Messages are sent and received between objects having certain functions and knowledge to carry out processes. Messages may be generated in response to user instructions, for example, by a user activating an icon with a “mouse” pointer generating an event. Also, messages may be generated by an object in response to the receipt of a message. When one of the objects receives a message, the object carries out an operation (a message procedure) corresponding to the message and, if necessary, returns a result of the operation. Each object has a region where internal states (instance variables) of the object itself are stored and where the other objects are not allowed to access. One feature of the object oriented system is inheritance. For example, an object for drawing a “circle” on a display may inherit functions and knowledge from another object for drawing a “shape” on a display.

A programmer “programs” in an object oriented programming language by writing individual blocks of code each of which creates an object by defining its methods. A collection of such objects adapted to communicate with one another by means of messages comprises an object oriented program. Object oriented computer programming facilitates the modeling of interactive systems in that each component of the system can be modeled with an object, the behavior of each component being simulated by the methods of its corresponding object, and the interactions between components being simulated by messages transmitted between objects.

An operator may stimulate a collection of interrelated objects comprising an object oriented program by sending a message to one of the objects. The receipt of the message may cause the object to respond by carrying out predetermined functions which may include sending additional messages to one or more other objects. The other objects may in turn carry out additional functions in response to the messages they receive, including sending still more messages. In this manner, sequences of message and response may continue indefinitely or may come to an end when all messages have been responded to and no new messages are being sent. When modeling systems utilizing an object oriented language, a programmer need only think in terms of how each component of a modeled system responds to a stimulus and not in terms of the sequence of operations to be performed in response to some stimulus. Such sequence of operations naturally flows out of the interactions between the objects in response to the stimulus and need not be preordained by the programmer.

Although object oriented programming makes simulation of systems of interrelated components more intuitive, the operation of an object oriented program is often difficult to understand because the sequence of operations carried out by an object oriented program is usually not immediately apparent from a software listing as in the case for sequentially organized programs. Nor is it easy to determine how an object oriented program works through observation of the readily apparent manifestations of its operation. Most of the operations carried out by a computer in response to a program are “invisible” to an observer since only a relatively few steps in a program typically produce an observable computer output.

In the following description, several terms which are used frequently have specialized meanings in the present context. The term “API” relates to a set of computer instructions and associated data which can be activated directly or indirectly by the user. The terms “windowing environment,” “running in windows,” and “API-oriented operating system” are used to denote a computer user interface in which information is manipulated and displayed on a video display such as within bounded regions on a raster scanned video display. The terms “network,” “local area network,” “LAN,” “wide area network,” or “WAN” mean two or more computers which are connected in such a manner that messages may be transmitted between the computers. In such computer networks, typically one or more computers operate as a “server,” which run one or more applications capable of accepting requests from clients and giving responses accordingly (and which, optionally, may also include a server operating system on top of which the other programs/applications run). Servers can run on any computer including dedicated computers, which individually are also often referred to as “the server” and typically comprise—or have access to—processors, memory, large storage devices (such as, for example, hard disk drives) and, optionally, communication hardware to operate peripheral devices such as printers, webcams, or modems. Servers can also be configured for cloud computing, which is Internet-based computing where groups of remote servers are networked to allow for centralized data storage. Such cloud computing systems enable users to obtain online access to computer services and/or other resources despite such users' potentially diverse geographic locations. Servers may also comprise uninterruptible power supplies to insure against power failure, as well as hardware redundancy such as dual power supplies, RAID disk systems, and ECC memory, along with extensive pre-boot memory testing and verification systems.

Other computers, termed “workstations” or “clients,” provide a user interface so that users of computer networks can access the network resources, such as shared data files, common peripheral devices, and inter workstation communication. Users activate computer programs or network resources to create “processes” which include both the general operation of the computer program along with specific operating characteristics determined by input variables and its environment. Similar to a process is an agent (sometimes called an intelligent agent), which is a process that gathers information or performs some other service without user intervention and on some regular schedule. Typically, an agent, using parameters typically provided by the user, searches locations either on the host machine or at some other point on a network, gathers the information relevant to the purpose of the agent, and presents it to the user on a periodic basis. A “module” refers to a portion of a computer system and/or software program that carries out one or more specific functions and may be used alone or combined with other modules of the same system or program.

The term “desktop” means a specific user interface which presents a menu or display of APIs with associated settings for the user associated with the desktop. When the desktop accesses a network resource, which typically requires an application program to execute on the remote server, the desktop calls an Application Program Interface, or “API” to allow the user to provide commands to the network resource and observe any output. The term “Browser” refers to a program which is not necessarily apparent to the user, but which is responsible for transmitting messages between the desktop and the network server and for displaying and interacting with the network user. Browsers are designed to utilize a communications protocol for transmission of text and graphic information over a worldwide network of computers, namely the “World Wide Web” or simply the “Web.” Examples of Browsers compatible with one or more embodiments described in the present application include, but are not limited to, the Chrome browser program developed by Google Inc. of Mountain View, Calif. (Chrome is a trademark of Google Inc.), the Safari browser program developed by Apple Inc. of Cupertino, Calif. (Safari is a registered trademark of Apple Inc.), Internet Explorer program developed by Microsoft Corporation (Internet Explorer is a trademark of Microsoft Corporation), the Opera browser program created by Opera Software ASA, or the Firefox browser program distributed by the Mozilla Foundation (Firefox is a registered trademark of the Mozilla Foundation). Although the following description details operations in terms of a graphic user interface of a Browser, it will be understood that one or more embodiments disclosed in the present disclosure may be practiced with text based interfaces, voice or visually activated interfaces, or any other interfaces now or hereinafter developed that have many of the functions of a graphic based Browser.

Browsers display information which is formatted in a Standard Generalized Markup Language (“SGML”) or a HyperText Markup Language (“HTML”), both being scripting languages which embed non visual codes in a text document through the use of special ASCII text codes. Files in these formats may be easily transmitted across computer networks, including global information networks like the Internet, and allow the Browsers to display text, images, and play audio and video recordings. The Web utilizes these data file formats to conjunction with its communication protocol to transmit such information between servers and workstations. Browsers may also be programmed to display information provided in an eXtensible Markup Language (“XML”) file, with XML files being capable of use with several Document Type Definitions (“DTD”) and thus more general in nature than SGML or HTML. The XML file may be analogized to an API, as the data and the stylesheet formatting are separately contained (formatting may be thought of as methods of displaying information, thus an XML file has data and an associated method). Similarly, JavaScript Object Notation (JSON) may be used to convert between data file formats.

The terms “personal digital assistant” or “PDA,” as defined above, means any handheld, mobile device that combines computing, telephone, fax, e-mail and/or networking features. The terms “wireless wide area network” or “WWAN” mean a wireless network that serves as the medium for the transmission of data between a handheld device and a computer. The term “synchronization” means the exchanging of information between a first device (e.g., a handheld device) and a second device (e.g., a desktop computer), either via wires or wirelessly. Synchronization ensures that the data on both devices are identical (at least at the time of synchronization).

In wireless wide area networks, communication primarily occurs through the transmission of radio signals over analog, digital cellular or personal communications service (“PCS”) networks. Signals may also be transmitted through microwaves and other electromagnetic waves. At the present time, most wireless data communication takes place across cellular systems using second generation technology such as code-division multiple access (“CDMA”), time division multiple access (“TDMA”), the Global System for Mobile Communications (“GSM”), Third Generation (wideband or “3G”), Fourth Generation (broadband or “4G”, also referred to as 4G LTE, referring to a long-term evolution), personal digital cellular (“PDC”), or through packet-data technology over analog systems such as cellular digital packet data (CDPD”) used on the Advance Mobile Phone Service (“AMPS”).

The terms “wireless application protocol” or “WAP” mean a universal specification to facilitate the delivery and presentation of web-based data on handheld and mobile devices with small user interfaces. “Mobile Software” refers to the software operating system which allows for application programs to be implemented on a mobile device such as a mobile telephone, PDA, tablet, wearable or smartphone. Examples of Mobile Software are Java and Java ME (Java and JavaME are trademarks of Sun Microsystems, Inc. of Santa Clara, Calif.), BREW (BREW is a registered trademark of Qualcomm Incorporated of San Diego, Calif.), Windows Mobile (Windows is a registered trademark of Microsoft Corporation of Redmond, Wash.), Palm OS (Palm is a registered trademark of Palm, Inc. of Sunnyvale, Calif.), Symbian OS (Symbian is a registered trademark of Symbian Software Limited Corporation of London, United Kingdom), ANDROID OS (ANDROID is a registered trademark of Google, Inc. of Mountain View, Calif.), and iPhone OS (iPhone is a registered trademark of Apple, Inc. of Cupertino, Calif.), and Windows Phone 10 (or predecessor or successor models/versions of any the foregoing). “Mobile Apps” refers to software programs written for execution with Mobile Software.

In the following specification, the term “social network” may be used to refer to a multiple user computer software system that allows for relationships among and between users (individuals or members) and content assessable by the system. Generally, a social network is defined by the relationships among groups of individuals, and may include relationships ranging from casual and/or professional acquaintances to close familial bonds. In addition, members may be other entities that may be linked with individuals such as, for example, universities or educational institutes. The logical structure of a social network may be represented using a graph structure. Each node of the graph may correspond to a member of the social network, or content assessable by the social network. Edges connecting two nodes represent a relationship between two individuals. In addition, the degree of separation between any two nodes is defined as the minimum number of hops required to traverse the graph from one node to the other. A degree of separation between two members is a measure of relatedness between the two members.

Conventional examples of social networks include, but are not limited to, Facebook, Twitter, LinkedIn, Instagram, and other systems or platforms. The exact terminology of certain features, such as associations, fans, profiles, etc. may vary from social network to social network, although there are several functional features that are common to the various terms. Thus, a particular social network may have more or less of the common features described above. In terms of the following disclosure, the general use of the term “social network” encompasses a system that includes one or more of the foregoing features or their equivalents.

To aid in understanding the novel concepts presented herein, a brief overview of an exemplary system 300 of the present disclosure and its functionality will now be described, followed by more detailed descriptions of the components thereof and its underlying system architecture and computing environments.

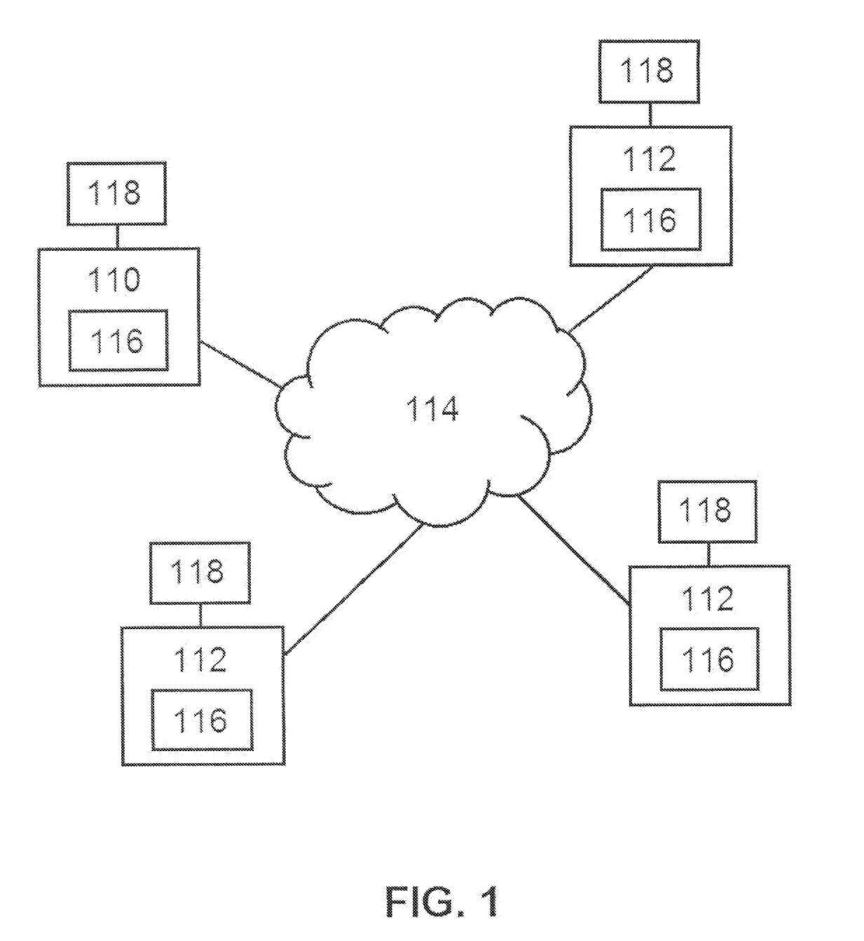

Now referring to the system architecture of the present disclosure, FIG. 1 is a high-level block diagram of a computing environment through which aspects of a presently disclosed system 300 and methods may be implemented. FIG. 1 illustrates a computing environment 100 (which may form at least part of an exemplary system 300 of the present disclosure) which comprises, for example, one server 110 and three clients 112 connected by network 114. The number of services 110 and clients 112 are not limited, as one or more servers 110 and/or one or more clients 112 may be used in various computing environments 100. One or more users 118, such as users or administrators (as described in further detail herein), can access the system via the one or more clients 112. Specifically, in at least one embodiment, the system 300 of the present disclosure is configured such that one or more users 118 can access the particular functionality of and/or data stored within the server 110 via a user interface (not shown in FIG. 1, but shown and described herein in connection with various system 300 embodiments) and the network 114. The computing environment may be configured similarly to a multi-user site in that numerous parties may register and/or access the server 110 via multiple—and commonly remote—clients 112. The server 110 is operatively coupled with the clients 112 over a network 114 or networking infrastructure. For example and without limitation, the network 114 may be operatively coupled with clients 112 via the Internet, intranet or other connection.

It will be appreciated that only three clients 112 are shown in FIG. 1 in order to simplify and clarify the description and the same is not intended to be limiting. Indeed, embodiments of the computing environment may have thousands or millions of clients 112 connected to network 114, for example the Internet. Likewise, while only one server 110 is depicted in FIG. 1, the computing environment may comprise a plurality of servers 110.

The clients 112 may each comprise one or more network accessible devices that are capable of executing one or more applications and/or accessing a web-based system through a browser. A client 112 may be any type of workstation such as, for example, any type of computer, computing device, or system of a type known in the art such as a personal computer, mainframe computer, workstation, notebook, tablet or laptop computer or device, PDA, mobile telephone or smartphone, wearable, or any other computing or communications device having network interfaces (wireless or otherwise). Additionally, users 118 may operate software 116 on one or more of clients 112 to both send and receive messages over the network 114 via server 110 and any of its associated communications equipment and software (not shown). Further, clients 112 may each comprise hardware and componentry as would occur to one of skill in the art such as, for example, one or more microprocessors, memory, input/output devices, device controllers, and the like. Clients 112 may also comprise one or more input devices that are operable by a user 118 of the client 112 such as, for example, a keyboard, keypad, pointing device, mouse, touchpad, touch screen, microphone, camera, webcam, and/or any other data entry means (or combination thereof) known in the art or hereinafter developed. Furthermore, client 112 may also comprise visual and/or audio display means for displaying or emitting output. For example, a client 112 may comprise a CRT display, an LCD display, a printer, one or more speakers, and/or any other types of display or output devices known in the art or hereinafter developed. The exact configuration of each client 112 in any particular implementation of a system 300 hereof may vary between clients 112 and may be left to the discretion of the practitioner.

As shown in FIG. 1, each client 112 is connected to, and/or in communication with, the server 110 via a network 114. The network 114 providing access to and/or serving as part of an exemplary system 300 of the present disclosure comprises any means for interconnecting the server 110 and a client 112. In at least one exemplary embodiment, the network 114 comprises the Internet, a global computer network. Alternatively, the network 114 may be selected from a variety of different networks and/or cables including, but not limited to, a commercial telephone network, one or more local area networks, one or more wide area networks, one or more wireless communications networks, coaxial cable(s), fiber optic cable(s), and/or twisted-pair cable(s). Additionally, the network 114 may comprise equivalents of any of the aforementioned, or combinations of two or more types of networks and/or cables.

Furthermore, in at least one embodiment where the server 110 and a client 112 comprise a single computing device operable to perform the functions delegated to both server 110 and a client 112 according to the present disclosure, the network 114 may comprise the hardware and software means interconnecting the server 110 and client 112 within the single computing device. Accordingly, the network 114 may comprise packet-switched facilities (such as the Internet), circuit-switched facilities (such as the public-switched telephone network), radio-based facilities (such as a wireless network), or any other facilities capable of interconnecting a client 112 with the server 110. Additionally, the clients 112 and/or wired/wireless connections may include the appropriate safeguards to ensure that the transmission of data between the server 110 and each client 112 is secure.

It will be appreciated that where the computing environment comprises a plurality of clients 112, such clients 112 need not all comprise the same type of client 112 or be in communication with the network 114 and/or server 110 via the same type of communication link. As such, the computing environment 100 may comprise some clients 112 configured to connect to/communicate with the server 110 via the Internet, for example, while other clients 112 are connected to the server 110 via a wired connection (e.g., a cable).

Exemplary systems 300 of the present disclosure may be implemented through any appropriate application architecture pattern now known or hereinafter developed. In at least one exemplary embodiment, the system 300 is delivered through an n-tier architecture in which presentation, application/business logic, and data management functions are logically and/or physically separated. This application architecture pattern provides benefits in the way of increasing availability of the system 300 to its users (i.e. reduced downtime), the minimization of the impact of any component failure, and through facilitating disaster recovery. Additionally, if desired, third party applications may be interfaced with the system 300 and provided to system users without sacrificing data security as such third party applications need not be in direct communication with the data structures of the system 300.

As described above, the clients 112 of the computing environment each comprise a user interface to facilitate a user's 118 input into and access to the functionality of and/or data stored within the server 110. The user interface can be any interface known in the art that is appropriate to achieve such a purpose and is fully customizable. For example, the display and content of the user interface may be customized for particular categories of users 118 such that the system 300 can provide standardized user interfaces having features and functionality that are specifically tailored to its different types of users' needs.

The user interface may be local to a client 112, provided over the network 114, or stored within the server 110. In at least one embodiment, the user interface comprises a web-based portal that provides functionality for accessing and displaying data stored within the server 110. In at least one exemplary embodiment, the user interface comprises a mobile application and/or widget designed to run on smartphones, tablet computers, wearables, and other mobile devices.

As previously described, one or more clients 112 can be operably connected to and/or in communication with the server 110 of the system 300's computing environment. In general, the system 300 supports at least two categories of users 118—administrators and individuals—that can logon and access system 300 via the client(s) 112. For the avoidance of doubt, when the term “user” is used herein, it shall mean an individual-user as opposed to an administrator, unless indicated otherwise.

The systems 300 of the present disclosure are not limited geographically. Users 118 from all over the world can participate, if desired, as there is no inherent restriction as to the number of users 118 who can access and use the system 300 at a single time.

In at least one embodiment, the system 300, or components thereof, is managed by one or more administrators, which may be individuals, business entities, and/or representatives of the foregoing. In this manner, the system 300 facilitates support of a substantial number of users 118. An administrator may have broad security credentials and/or access permissions that provide it, for example, with access to data stored in the system 300 and/or the ability to run and view data analytics.

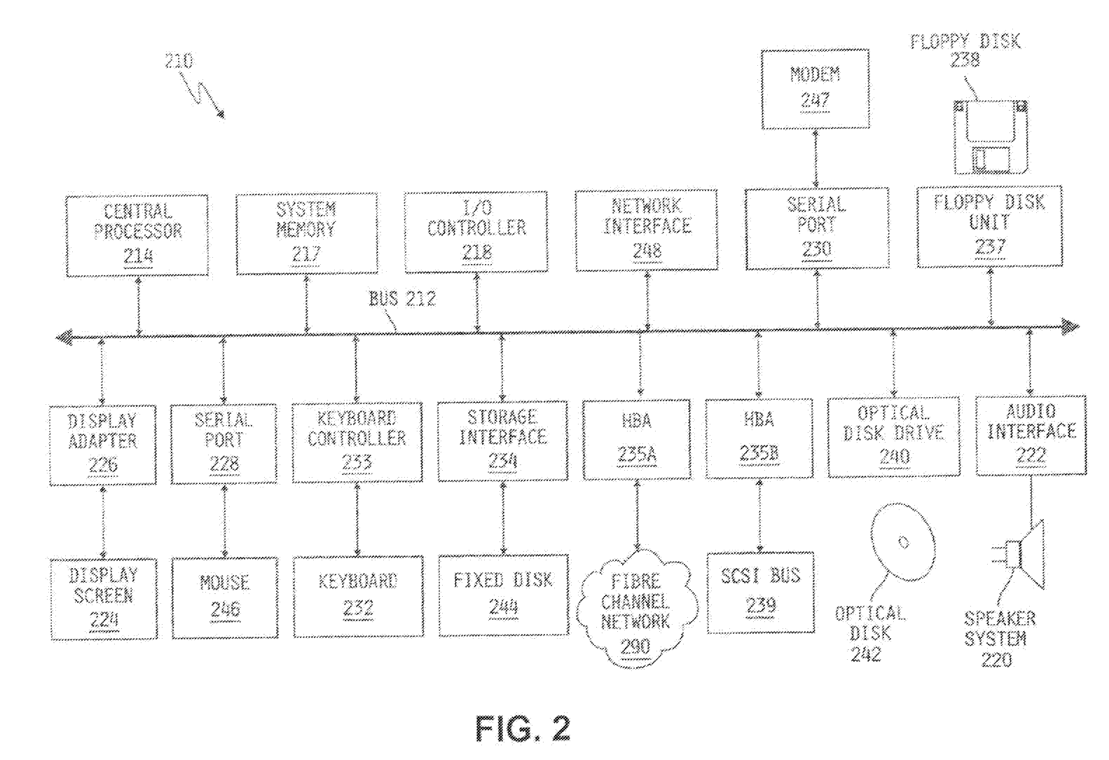

Now referring to FIG. 2, a block diagram of a computer system hardware 210 suitable for implementing the system 300 via server 110 or client 112 is shown. Exemplary computer systems 210 of the present disclosure include a bus 212 that interconnects major subsystems of computer system 210, such as a central processor 214 (also referred to generally as a “processor”), system memory 217 (typically RAM, but which may also include ROM, flash RAM, or the like and/or ECC memory and/or RAID disk systems), one or more input/output controllers 218, external audio devices (such as speaker system 220 via audio output interface 222), external devices (such as display screen 224 via display adapter 226), serial ports 228 and 230 (which can be, for example, traditional serial ports, parallel ports, universal serial bus (USB) ports, and the like), keyboards 232 (interfaced with keyboard controller 233), storage interfaces 234, disk drives 237 operative to receive floppy disk 238, host bus adapter (HBA) interface cards 235A operative to connect with fibre channel network 290, HBA interface cards 235B operative to connect to SCSI busses 239, and optical disk drives 240 operative to receive optical disk 242, for example. Various computer systems 210 may include one or more of some or all of the foregoing, or similar hardware and/or software as may be hereinafter developed. Also included, for example, are mouse 246 (or other point-and-click devices or other input devices, such as touchpads or touchscreens coupled to bus 212 via serial port 228), modem 247 (coupled to bus 212 via serial port 230), and network interface 248 (coupled directly to bus 212).

Bus 212 allows data communication between central processor 214 and system memory 217, which may include read-only memory (ROM) or flash memory, random access memory (RAM), RAID (redundant array of independent disks) disk systems, ECC memory (error-correcting code memory), or any combination of the foregoing as previously noted (the foregoing examples not specifically shown). RAM is generally the main memory into which operating system and application programs are loaded. ROM or flash memory may contain, among other software code, Basic Input-Output system (BIOS) which controls basic hardware operation such as interaction with peripheral components. Applications resident with computer system 210 are generally stored on and accessed via computer readable media, such as hard disk drives (e.g., fixed disk 244), optical drives (e.g., optical drive 240), floppy disk unit 237, or other storage media known or developed in the art (referred to as a storage medium or multiple storage media). Additionally, applications may be in the form of electronic signals modulated in accordance with the application and data communication technology when accessed via network modem 247 or interface 248 or other telecommunications equipment (not shown).

Storage interface 234, as with other storage interfaces of computer system 210, may connect to standard computer readable media for storage and/or retrieval of information, such as fixed disk drive 244. Fixed disk drive 244 may be part of computer system 210 or may be separate and accessed through other interface systems. Modem 247 may provide direct connection to remote servers via telephone link or the Internet via an internet service provider (ISP) (not shown), or as is otherwise available. Network interface 248 may provide direct connection to remote servers via direct network link to the Internet via a POP (point of presence). Network interface 248 may provide such connection using wireless techniques, including digital cellular telephone connection, Cellular Digital Packet Data (CDPD) connection, digital satellite data connection or the like.

Many other devices or subsystems (not shown) may be connected in a similar manner (e.g., document scanners, digital cameras and so on). Conversely, all of the devices shown in FIG. 2 need not be present to practice the present disclosure. Furthermore, devices and subsystems may be interconnected in different ways from that shown in FIG. 2. Operation of a computer system such as that shown in FIG. 2 is readily known in the art and is not discussed in detail in this application. Software source and/or API specifications to implement the present disclosure may be stored in computer-readable storage media such as one or more of system memory 217, fixed disk 244, optical disk 242, or floppy disk 238. The operating system provided on computer system 210 may be a variety or version of either MS-DOS® (MS-DOS is a registered trademark of Microsoft Corporation of Redmond, Wash.), WINDOWS® (WINDOWS is a registered trademark of Microsoft Corporation of Redmond, Wash.), OS/2® (OS/2 is a registered trademark of International Business Machines Corporation of Armonk, N.Y.), UNIX® (UNIX is a registered trademark of X/Open Company Limited of Reading, United Kingdom), Linux® (Linux is a registered trademark of Linus Torvalds of Portland, Oreg.), various Apple® operating systems (OSs), or other known or developed operating system. In some embodiments, computer system 210 may take the form of a tablet computer, typically in the form of a large display screen operated by touching the screen. In tablet computer alternative embodiments, the operating system may be iOS® (iOS is a registered trademark of Cisco Systems, Inc. of San Jose, Calif., used under license by Apple Corporation of Cupertino, Calif.), Android® (Android is a trademark of Google Inc. of Mountain View, Calif.), Blackberry® Tablet OS (Blackberry is a registered trademark of Research In Motion of Waterloo, Ontario, Canada), webOS (webOS is a trademark of Hewlett-Packard Development Company, L.P. of Texas), and/or other suitable tablet operating systems.

Moreover, regarding the signals described herein, those skilled in the art will recognize that a signal may be directly transmitted from a first block to a second block, or a signal may be modified (e.g., amplified, attenuated, delayed, latched, buffered, inverted, filtered, or otherwise modified) between blocks. Although the signals of the certain embodiments described herein are characterized as transmitted from one block to the next, other embodiments of the present disclosure may include modified signals in place of such directly transmitted signals as long as the informational and/or functional aspect of the signal is transmitted between blocks. To some extent, a signal input at a second block may be conceptualized as a second signal derived from a first signal output from a first block due to physical limitations of the circuitry involved (e.g., there will inevitably be some attenuation and delay). Therefore, as used herein, a second signal derived from a first signal includes the first signal or any modifications to the first signal, whether due to circuit limitations or due to passage through other circuit elements which do not change the informational and/or final functional aspect of the first signal.

The various feature sets of systems 300 will now be described in detail, which may include screenshots of embodiments of graphical user interfaces (“GUIs”, also referred to herein as user interfaces) for explanatory purposes in some cases. It will be understood that these GUIs are simply examples of various embodiments of the system 300 and are not intended to be limiting in any manner. Indeed, unless otherwise expressly stated herein, it will be understood that the specific GUIs used with a system 300 are fully customizable in accordance with the requirements and desires of a user.

It is noted that various system 300 embodiments of the present disclosure may use various components of an exemplary computer system 210 as referenced herein and shown in FIG. 2, such as, for example, a central processor 214 (or generally one or more processors), storage media (such as, for example, one or more hard disk drives (e.g., fixed disks 244), one or more optical drives (e.g., optical drive 240), one or more floppy disk units 237, etc.), etc. Processor(s) 214 would then be operable and/or configured to perform or otherwise carry out one or more of the various processes or method steps referenced herein, such as by way of executing instructions within software 116, so to generally operate aspects of system 300. Processor(s) 214 also generally control the access to and use of various data so to create results, perform tasks, calculate items, transform data into something else (such as a data-based result), whereby, for example, the various results (exemplary outputs of system 300) could be used to control the operation and/or activity of processor(s) 214 to perform additional tasks, for example.

Systems 300 of the present disclosure can comprise/include data relating to one or more of the following, by way of example:

1. Computerized Tomography (CT)

2. Magnetic Resonance Imaging (MRI)

3. Positron Emission Tomography (PET)

4. Ultrasound

5. Other bodily imaging (that is not CT, MRI, PET, or ultrasound)

For example, and in at least one system 300 embodiment, system 300 uses a plurality of computational simulations, such as a first computational simulation 302, a second computational simulation 304, and a third computational simulation 306. Additional computation simulations could also be used by system 300, as described in further detail herein. Said computational simulations, such as computational simulations 302, 304, 306, etc., can comprise computational simulations within a library 310. Library 310, therefore, would include at least two computational simulations, and in at least some embodiments more than two, such as simulations 302, 304, 306, etc.

Computational simulations 302, 304, 306, etc., as referenced herein, may include any number of the following, by way of example.

a. Cardiovascular simulations, such as those relating to vascular geometry and movement during the cardiac cycle

b. Cardiac simulations, such as those relating to cardiac geometry and movement during the cardiac cycle

c. Valve simulations, such as aortic valve simulations

d. Blood flow simulations, such as flow rate and/or flow quantity

e. Stent and/or stent graft simulations

f. Cardiac mechanics simulations to compute the stress in the wall of the heart which determines the metabolic requirements of the heart cells

g. Simulations of blood flow in the ventricles of the heart to determine blood pressure distribution (e.g., non-invasive measurement of pressure in the right atrium or ventricle or pulmonary artery).

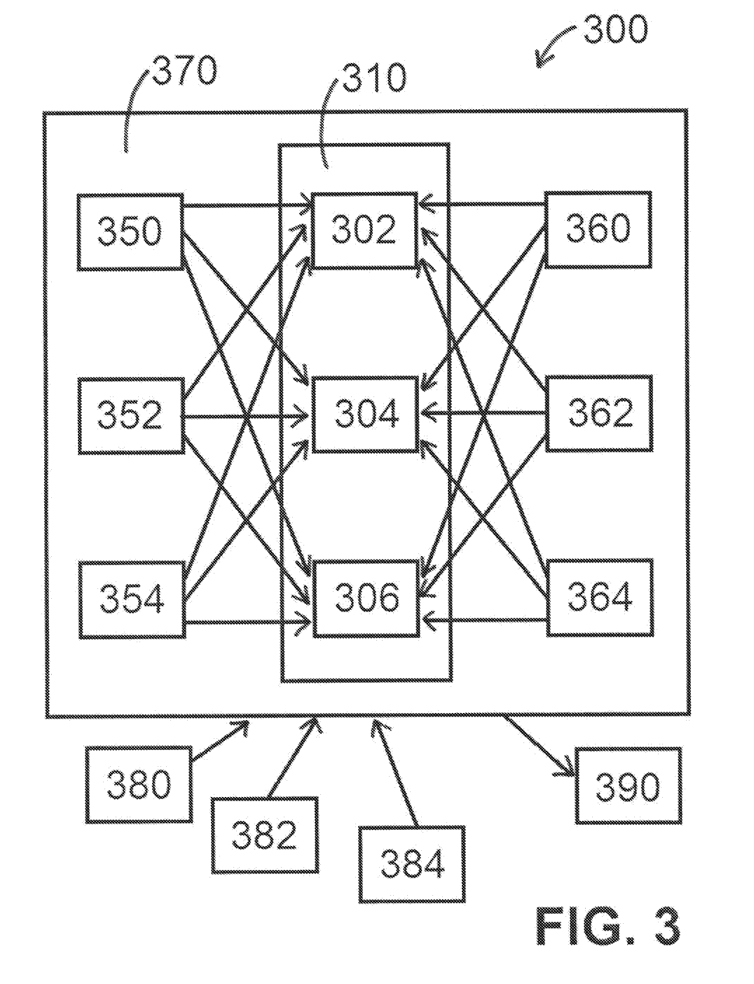

The aforementioned computational simulations 302, 304, 306, etc. (also referred to herein as patient simulations 302, 304, 306, etc.), are generated using generic information and patient information. For example, any number of patient simulations 302, 304, 306, etc., could be generated using a combination of any of first generic information 350, second generic information 352, third generic information 354, additional generic information, and any of first model patient information 360, second model patient information 362, third model patient information 364, and/or additional model patient information. As shown in FIG. 3, for example, said computational simulations 302, 304, 306 comprising any number of generic information 350, 352, 354 and any number of model patient information 360, 362, 364, can collectively form combined simulation 370.

Generic information 350, 352, 354, etc., can comprise, for example, field equations (laws of mechanics, conservation of mass, momentum, and/or energy), such as those using a geometric mesh on which the computations are performed, for example, including but not limited to information that is generic to the model/simulation. Model patient information 360, 362, 364, etc., can comprise, for example patient-specific geometry or structure of an organ of interest. For example, and for a blood vessel, it could be the geometry of the vessel, and for the heart, it could be the geometry of left or right ventricles, etc. Said model patient information 360, 362, 364, etc., can also involve material properties or mechanical properties of the patient-specific system (i.e., stress-strain relation), and/or use boundary or loading conditions; e.g., patient blood pressure in the vessel or ventricle.

Combined simulation 370, as shown in FIG. 3, or one or more individual patient simulations 302, 304, 306, etc., can be used as follows. A patient of interest, as referenced herein, can be evaluated, tested, analyzed, etc., so that one or more patient-specific parameters can be obtained, such as a first patient-specific parameter 380, a second patient-specific parameter 382, a third patient-specific parameter 384, etc., which are therefore specific to the patient of interest, such as, for example, any number of the anatomy, material properties, and/or boundary conditions referenced herein. One or more of the patient-specific parameters 380, 382, 384 can be analyzed in view of any of computation simulations 302, 304, 306, etc., or combined simulation 370, such as shown in FIG. 3, so to generate patient-specific outcome 390. Patient-specific outcome 390, such as shown in FIG. 5, can comprise one or more outcome elements, such as a first outcome element 450, a second outcome element 452, a third outcome element 454, etc., whereby said outcome elements 450, 452, 454 each represent characteristics of the patient of interest as relating to the application of the one or more patient-specific parameters 380, 382, 384 to combined simulation 370 or one or more individual patient simulations 302, 304, 306, etc.

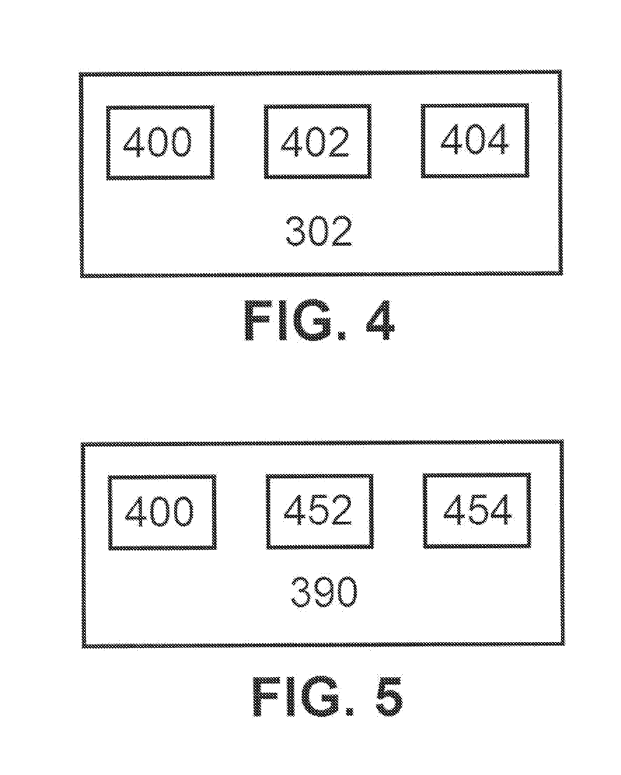

FIG. 4 shows a block diagram of how an exemplary patient simulation (shown as patient simulation 302 for discussion purposes) can have individual aspects depending on the patient of interest. For example, patient simulation 302 can have a first aspect 400, a second aspect 402, a third aspect 404, etc., whereby said aspects 400, 402, 404, etc., are variations of patient simulation 302 depending on one or more parameters, such as, for example, age, weight, medical history, and the like. By way of example, aspects 400, 402, 404 could relate to age, whereby aspect 400 is a first age or age range, aspect 402 is a second age or age range, and aspect 404 is a third age or age range, each differing from one another. If the patient of interest has an age of the first age or falling within the age range of aspect 400, aspect 400 of simulation 302 could be used individually, or along with other aspects 402, 404, etc. and/or other simulations 302, 304, etc. as part of a combined simulation 370, as noted above.

As referenced herein, the present disclosure includes disclosure of the construction of a library of patient-specific simulations, referred to as a combined simulation 370. Based on general population parameters of anatomy and physiology, numerous mathematical models can be constructed (individual patient simulations 302, 304, 306, etc.) that vary within the range of population parameters. The simulations 302, 304, 306 can be executed and catalogued for real-time access based on the input parameters that fit closest to the patient of interest. Since the parameters will be discrete at the outset and may not represent exactly the patient of interest, mathematical interpolations and optimizations (e.g., non-linear least square fits) can be used to find the models that are closest to the patient of interest. The new patient data can then be executed and included in the library of models data bases to enable machine learning with the expansion of data base. This would allow continuous enhancements of the data bases to provide nearly continuous parameter space for real-time access of previously competed simulations.

Said systems 300 of the present disclosure can allow real-time extrapolation of simulation results (patient-specific outcomes 390) specific to a patient of interest to guide therapy to allow clinicians to optimize therapy based on evidence-based medicine.

Such an approach formalizes precision medicine that takes a quantitative approach to evidence-based medicine whereby the accumulation of clinical experience can be used to improve treatment of various diseases. For example, a patient that requires bypass surgery can have a virtual procedure that optimizes the graft placement (location, angle of anastomosis, length of graft, etc.) tailored to the patient's coronary anatomy, heart function, etc.

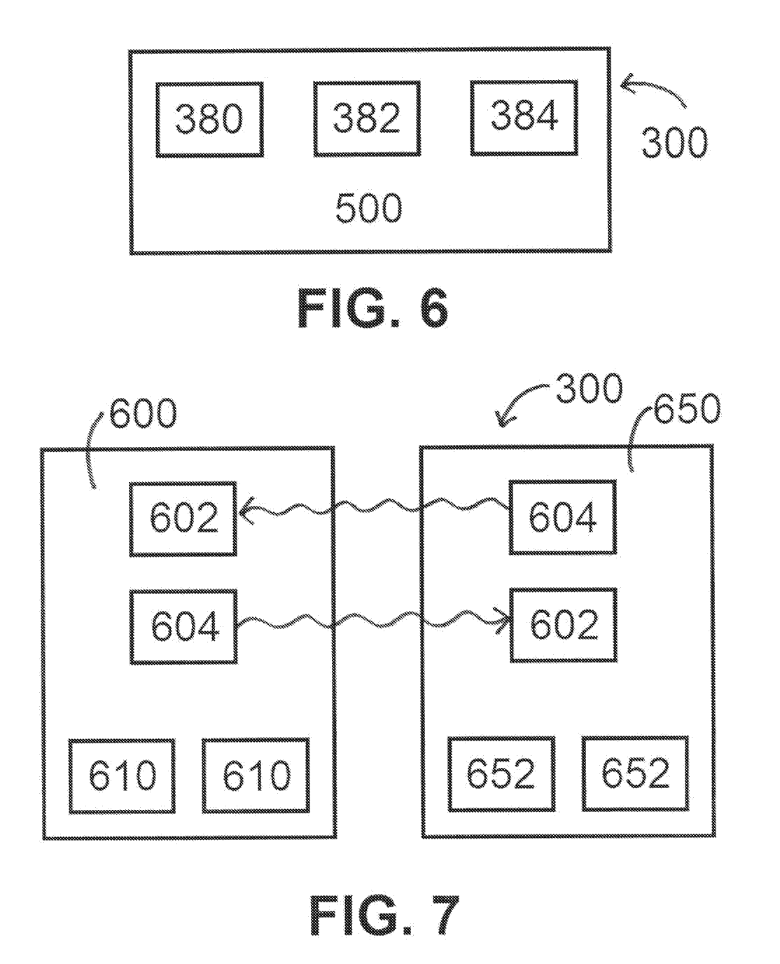

In at least one embodiment, an exemplary electronic medical record (EMR) 500 of a patient of interest, such as shown in FIG. 6, can comprise, for example, a first patient-specific parameter 380, a second patient-specific parameter 382, a third patient-specific parameter 384, or more patient-specific parameters. Said patient-specific parameters 380, 382, 384, etc., may comprise, for example, any number of parameters indicative of the patient of interest including, but not limited to, heart rate (pulse), blood pressure, blood glucose levels, cholesterol levels, etc. Said patient-specific parameters 380, 382, 384, etc., can be historical, such as heart rate (pulse), blood pressure, blood glucose levels, cholesterol levels, weight, current and/or past medications, prior medical procedures, diagnosed diseases, etc., and/or can be current, such as heart rate (pulse), blood pressure, blood glucose levels, cholesterol levels, etc., obtained using an exemplary system 300 of the present disclosure, as referenced in further detail herein. Patient-specific parameters 380, 382, 384, etc., can be obtained by various devices, such as referenced herein, and/or inputted directly by a user, also as referenced herein.

Said medical record 500, containing prior and/or current information/data (patient-specific parameters 380, 382, 384, etc., can be used to establish a real-time assessment and display of risk factors on a device 600 that monitors heart rate, blood pressure (see below), glucose or cholesterol levels, etc., such as by using various sensors. As shown in FIG. 7, an exemplary system 300 of the present disclosure comprises a first device 600, configured as exemplary system hardware 210, a server 110, or a client 112 of the present disclosure. Exemplary first devices 600 of the present disclosure may comprise a smartphone, namely a handheld device configured to run various applications (or “apps,” namely exemplary software 116 of the present disclosure) configured to run on exemplary system hardware 210, such as a client 112 configured as a smartphone. First device 600 (such as a smartphone), such as shown in FIG. 7, is configured to receive data/information from a remote device 650, such as by way of a receiver 602, and, in certain embodiments, is also configured to transmit data/information to a remote device 650, such as by way of a transmitter 604. In some embodiments, receiver 602 is also configured as a transmitter so to transmit data/information, and transmitter 604 is also configured as a receiver so to receive data/information. Transmission of data/information is shown in FIG. 7 by way of wavy directional arrows.

Remote devices 650, for example, can be configured as exemplary system hardware 210, a server 110, or a client 112 of the present disclosure. Remote devices 650, for example, can be configured as or having/comprising one or more sensors, electrodes, etc. For example, remote devices 650 can comprise one or more sensors or electrodes 652 configured as heart rate sensors (to obtain heart rate data), blood pressure sensors (to obtain blood pressure data), blood glucose sensors (to obtain blood glucose data), temperature sensors (to obtain bodily temperature data), blood alcohol level sensors (to obtain bodily blood alcohol level data), drug detection sensors (to obtain bodily drug level data) etc., so that remote devices 650, when used by a user (such as positioned upon or at least partially within the body) wears or otherwise uses such a remote device 650, the remote device 650 can obtain the desired bodily data/information that can be transmitted to first device 600 so that the user or wearer of first device 600 has ready access to said data/information.

Remote devices 650 can comprise a transmitter 604, such as shown in FIG. 7 configured to transmit data/information from remote devices 650 to first devices 600, as referenced herein. Remove devices 650 can comprise a receiver 602 configured to receive data/information from first device 600 (such as from transmitter 604 of first device) and/or from one or more sensors or electrodes 652. Exemplary remote devices 650 of the present disclosure can be configured as a smartwatch, namely a wrist-worn device configured to run various applications (or “apps,” namely exemplary software 116 of the present disclosure) configured to run on exemplary system hardware 210, such as a client 112 configured as a smartwatch.

Remote devices 650 can be configured as patch devices, such as adhesive patch devices configured to be affixed to the skin. Remote devices 650 can also be configured as sensors or electrodes positioned upon or within the body, as referenced above.

The data/information obtained from sensors or electrodes 652 and transmitted to one or more first devices 600 from remote device(s) 650 can be stored within remote device(s) 650, within first device(s) 600, or elsewhere, such as within various types of storage media referenced herein and/or known or used in the art. Medical records 500 of the present disclosure can therefore include some of said data/information obtained from sensors or electrodes 652 and transmitted to one or more first devices 600, and can be stored within remote device(s) 650, within first device(s) 600, or elsewhere, such as within various types of storage media referenced herein and/or known or used in the art.

Medical records 500 of the present disclosure can comprise various patient-specific parameters 380, 382, 384, etc., as referenced herein, whereby said patient-specific parameters 380, 382, 384, etc. comprise information/data obtained from sensors or electrodes 652 of remote devices 650, as referenced above. Medical records 500 of the present disclosure can also comprise various patient-specific parameters 380, 382, 384, etc., as referenced herein, whereby said patient-specific parameters 380, 382, 384, etc. comprise information/data obtained from sensors or electrodes 610 of first devices 600, as referenced further herein.

FIG. 7 shows an exemplary first device 600 comprising two sensors or electrodes 610, noting that one, two, three, or more sensors or electrodes 610 could comprise part of first device 600. In such an embodiment, first device 600 can obtain data/information from the body by way of operation of sensors or electrodes 610 configured as heart rate sensors (to obtain heart rate data), blood pressure sensors (to obtain blood pressure data), blood glucose sensors (to obtain blood glucose data), etc. as referenced herein, so that remote devices 650, when worn by a user (such as a smartwatch, for example), can obtain the desired bodily data/information so that the user or wearer of first device 600 has ready access to said data/information. In at least one system 300 embodiment, data/information can be obtained from sensors or electrodes 652 of remote device 650 and transmitted to first device 600 for ready access by a user, whereby said data/information comprises at least part of the various patient-specific parameters 380, 382, 384, etc., of medical record 500. In at least another system 300 embodiment, data/information can be obtained from sensors or electrodes 610 of first device 600 as well, whereby said data/information also comprises at least part of the various patient-specific parameters 380, 382, 384, etc., of medical record 500. For example, a first device 600 configured as a smartwatch could also obtain pulse data by way of operation of a sensor or electrode 610 of first device configured to obtain said data, and remote device 650 positioned upon or within the body could obtain other data/information from the body, whereby the data/information from sensors or electrodes 610, 652 could form at least part of the various patient-specific parameters 380, 382, 384, etc., of medical record 500.

Various patient-specific parameters 380, 382, 384, etc., of medical record 500 can also be directly inputted by a user, as referenced above. For example, patient-specific parameters 380, 382, 384, etc., of medical record 500 can be inputted by patient such as smoking data/information, alcohol consumption data/information, and/or drug consumption data/information, etc. Environmental risk data/information, such as level of smog, etc. can also be inputted by patient. This data/information could form part of the user's medical record 500.

Exemplary systems 300 of the present disclosure can therefore comprises one or more first devices 600, one or more second devices 650, and one or more medical records 500, as referenced herein.

Systems 300 of the present disclosure have important utility, namely to provide a real-time assessment of risk factors that provides medical guidelines for reduction of risk factors and hence prevention of disease. Prevention is key to maintenance of health and reduction of health care costs. Any prediction of predilection of disease can enable early intervention. This philosophy is the pillar of the proposed approach.

As the user's medical record 500 is updated with information/data, such as information/data directly inputted by the user or information/data obtained from sensors or electrodes 610 and/or 652, said medical record 500 can provide the user with information regarding potential risk factors that can be acted upon by the user as desired. For example, should data/information relating to pulse, blood pressure, temperature, cholesterol, etc., change over time in a way that would cause the user to exhibit a risk factor for a potential disease or condition that the user was not previously at risk for, system 300 could alert the user to said risk factor. For example, if a user of system 300 has an increase in blood pressure, as indicated by data/information obtained by sensors or electrodes 610, 652, and said increase in blood pressure is potentially indicative of hypertension, heart disease, etc., system 300 could alert the user by way of an indication on first device 600 readily accessible by said user.

Exemplary medical records 500 of the present disclosure can also include medical simulation data, such as data based upon one or more computational simulations, generic information, and/or model patient information, as may be desired.

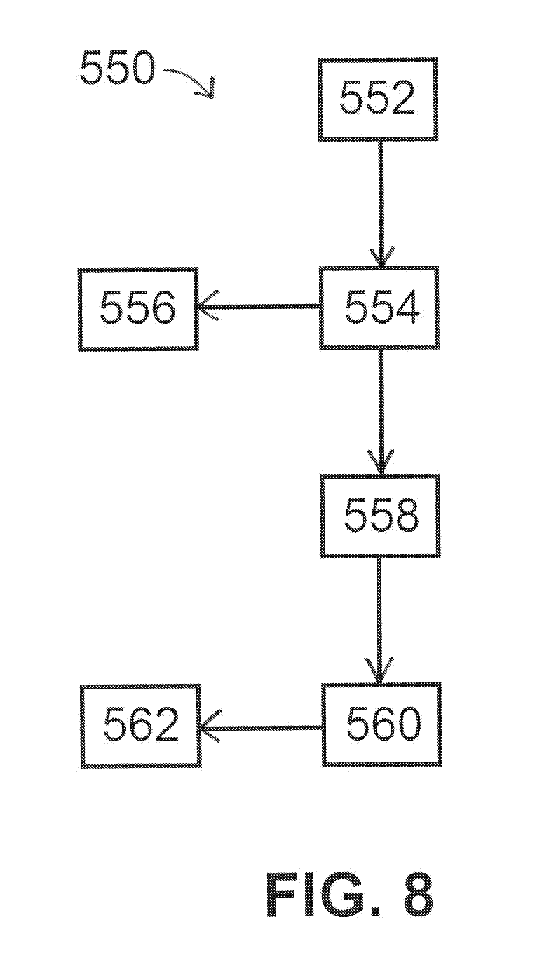

Said feedback is illustrated in FIG. 8. For example, an exemplary feedback loop (method 550) of the present disclosure includes the step of obtaining initial medical record 500 data (initial obtaining step 552), whereby some patient-specific parameters 380, 382, 384, etc., of medical record 500 are obtained and accessible by portions of system 300. A user risk factor baseline can then be generated (baseline generation step 554) by system 300 based upon said initial medical record 500, so that potential changes in risk factors can be determined in the future. Should performance of baseline generation step 554 indicate one or more risk factors, said risk factors can be reported/provided to the user by way of initial risk factor reporting step 556. Method 550 can further comprise the step of obtaining additional data/information (additional data step 558), whereby said additional data/information is directly inputted into system 300 by a user and/or is obtained from sensors or electrodes 610, 652. Said additional data/information can then be added to the initial medical record 500 (or the then-current medical record 500) so that a then-current risk factor level can be generated (current risk factor level generation step 560) based upon the then-available medical record 500. Should a change in the risk factors from the baseline generation step 554 be made from current risk factor level generation step 560, such as the removal and/or addition of one or more risk factors, said change in the risk factors can be reported to the user by way of current risk factor reporting step 562. Method 550 can then perform additional data step 558 over time, such as by obtaining additional data/information from a user and/or from sensors or electrodes 610, 652, as indicated by the directional arrow from step 560 to step 558. This loop can be repeated over time to provide real-time risk factor information to the user. Said method 550 can be facilitated/performed using an “app,” or software 116, as referenced herein.

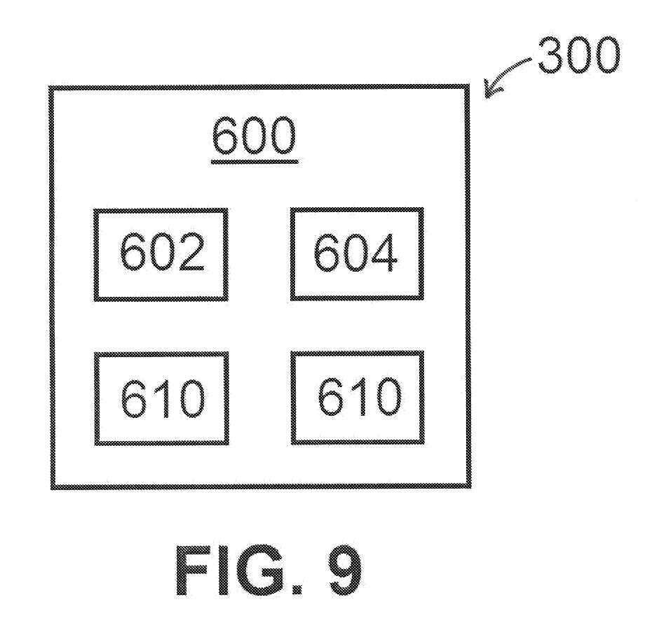

FIG. 9 shows an exemplary system 300 of the present disclosure. An exemplary system 300 of the present disclosure may comprise a first device 600. Exemplary first devices 600 of the present disclosure may comprise a smartphone, namely a handheld device configured to run various applications (or “apps,” namely exemplary software 116 of the present disclosure) configured to run on exemplary system hardware 210, such as a client 112 configured as a smartphone. Exemplary first devices 600 (such as a smartphones), such as shown in FIG. 9, may comprise one or more sensors or electrodes 610, noting that one, two, three, or more sensors or electrodes 610 could comprise part of first device 600. Exemplary first devices 600 of the present disclosure can have any number of components or features, such as a client 112 of the present disclosure, including, but not limited to, a wired or wireless receiver 602 and/or wireless transmitter 604, such as shown in FIG. 9.

Sensor(s) or electrode(s) 610, in at least some embodiments of the present disclosure, are configured to detect sound vibrations and/or turbulence created by stenosis or constriction of vessel lumen. Blood flow through a vessel with no obstruction will not produce audible sounds. The presence of a stenosis, however, will produce flow disturbances and hence pressure fluctuations in the form of sound waves. Placement of a sensor or electrode 610, such as a strain gauge (e.g., strain gauge) will detect sound fluctuations that can be recorded by a phone app (software 116). A library 310 of models (referenced in further detail herein) can then be accessed to produce a model with a degree of stenosis that corresponds to the produced flow disturbances or sound fluctuations. The solution of these problems follows the field equations of fluid mechanics (i.e., Navier-Stokes equations).

As referenced above, a first device 600 can obtain data/information from the body, such as to detect sound vibrations and/or turbulence created by stenosis or constriction of a vessel lumen, by way of operation of sensors or electrodes 610 configured to obtain said sound vibration and/or turbulence data. Said sound vibration and/or turbulence data, obtained by first device 600 (such a smartphone), could be recorded by an app (software 116) on said first device 600. Said data could then be processed by system 300, as follows.

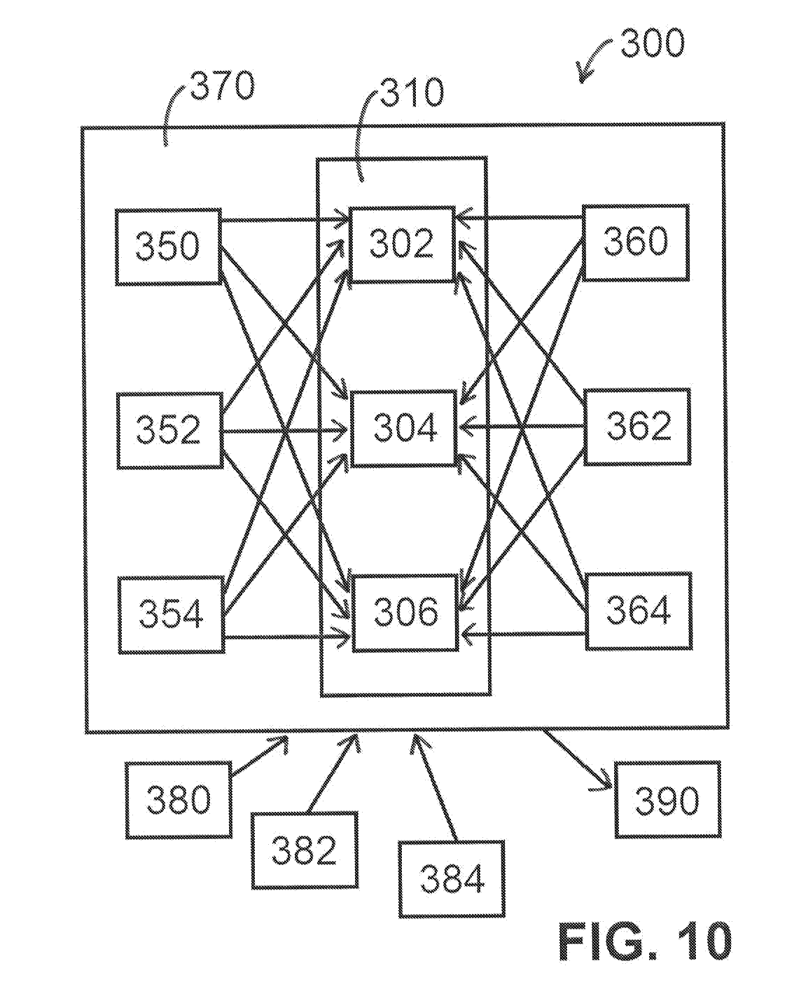

In at least one system 300 embodiment, such as shown in FIG. 10, system 300 uses a plurality of computational simulations, such as a first computational simulation 302, a second computational simulation 304, and a third computational simulation 306. Additional computation simulations could also be used by system 300, as described in further detail herein. Said computational simulations, such as computational simulations 302, 304, 306, etc., can comprise computational simulations within a library 310. Library 310, therefore, would include at least two computational simulations, and in at least some embodiments more than two, such as simulations 302, 304, 306, etc. Computational simulations 302, 304, 306, etc., as referenced herein, may include any number of simulations, including those relating to blood flow, vessel geometry, simulations of blood flow, and the like.

The aforementioned computational simulations 302, 304, 306, etc., are generated using generic information and patient information. For example, any number of patient simulations 302, 304, 306, etc., could be generated using a combination of any of first generic information 350, second generic information 352, third generic information 354, additional generic information, and any of first model patient information 360, second model patient information 362, third model patient information 364, and/or additional model patient information. As shown in FIG. 10, for example, said computational simulations 302, 304, 306 comprising any number of generic information 350, 352, 354 and any number of model patient information 360, 362, 364, can collectively form combined simulation 370.

Generic information 350, 352, 354, etc., can comprise, for example, field equations (laws of mechanics, conservation of mass, momentum, and/or energy), such as those using a geometric mesh on which the computations are performed, for example, including but not limited to information that is generic to the model/simulation. Model patient information 360, 362, 364, etc., can comprise, for example patient-specific geometry or structure of organ of interest. For example, and for a blood vessel, it could be the geometry of the vessel, and for the heart, it could be the geometry of left or right ventricles, etc. Said model patient information 360, 362, 364, etc., can also involve material properties or mechanical properties of the patient-specific system (i.e., stress-strain relation), and/or use boundary or loading conditions; e.g., patient blood pressure in the vessel or ventricle.

Combined simulation 370, as shown in FIG. 10, or one or more individual patient simulations 302, 304, 306, etc., can be used as follows. A patient of interest, as referenced herein, can be evaluated, tested, analyzed, etc., so that one or more patient-specific parameters can be obtained, such as a first patient-specific parameter 380, a second patient-specific parameter 382, a third patient-specific parameter 384, etc., which are therefore specific to the patient of interest, such as, for example, any number of the anatomy, material properties, and/or boundary conditions referenced herein. The vibration and/or turbulence data obtained using first device 600, for example, could be one or more of these parameters 380, 382, 384, etc., and processed according to the present disclosure, as may be desired. One or more of the patient-specific parameters 380, 382, 384 can be analyzed in view of any of computation simulations 302, 304, 306, etc., or combined simulation 370, such as shown in FIG. 10, so to generate patient-specific outcome 390.

An assessment of the degree of carotid stenosis can provide early detection of disease to improve outcome. Consistent with the foregoing, an early suggestion of stenosis will alert the patient to seek medical consultations or other attention. Confirmation of disease with imaging would enable early management of disease. This approach enables the patient to take an active role in their health in partnership with their physician.

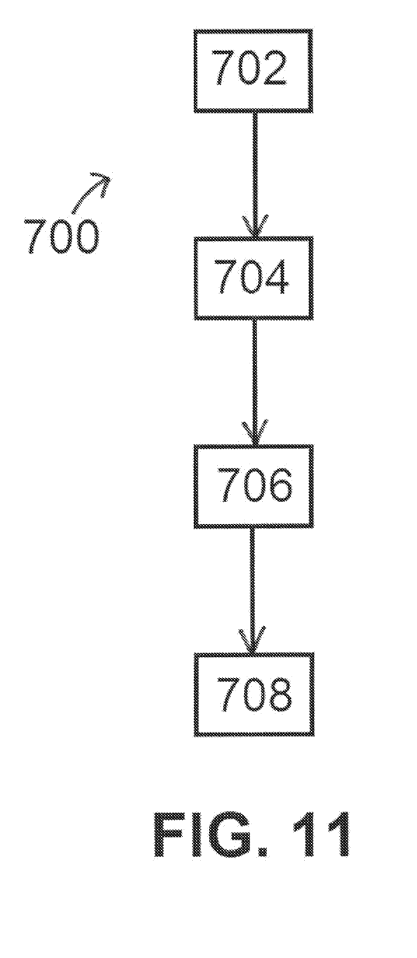

An exemplary method 700 of the present disclosure is shown in block step format in FIG. 11. As shown in FIG. 11, an exemplary method 700 of the present disclosure includes the step of operating a smartphone (an exemplary first device 600) having an app (software 116) stored thereon or accessibly therefrom, whereby the smartphone (first device 600) has one or more sensors or electrodes 610 that are operated to obtain vibration and/or turbulence data from the user of first device 600 (an exemplary operation step 702). Method 700 may further comprise the step of comparing the vibration and/or turbulence data with other information within a library 310 or to prior vibration and/or turbulence data (an exemplary data processing step 704) to obtain a comparison result, and should any (or a subset of) the vibration and/or turbulence data (including that within the comparison result) indicate a carotid stenosis or a degree of a carotid stenosis near, at, or above a threshold stenosis level, alerting the user of the first device 600, whereby the alert is provided by first device 600 itself (an exemplary alerting step 706). Should the user be alerted by step 706, such as by some sort of visual and/or auditory signal provided by first device 600, the user could then seek medical attention and potential treatment (an exemplary medical attention step 708).

In view of the foregoing, the present disclosure includes disclosure of devices and systems configured to assess a carotid stenosis, alert the user of said devices or systems of the same, to that the user can seek medical attention so to alter the progression of the stenosis and potentially prevent and/or alleviate the negative effects of stroke.

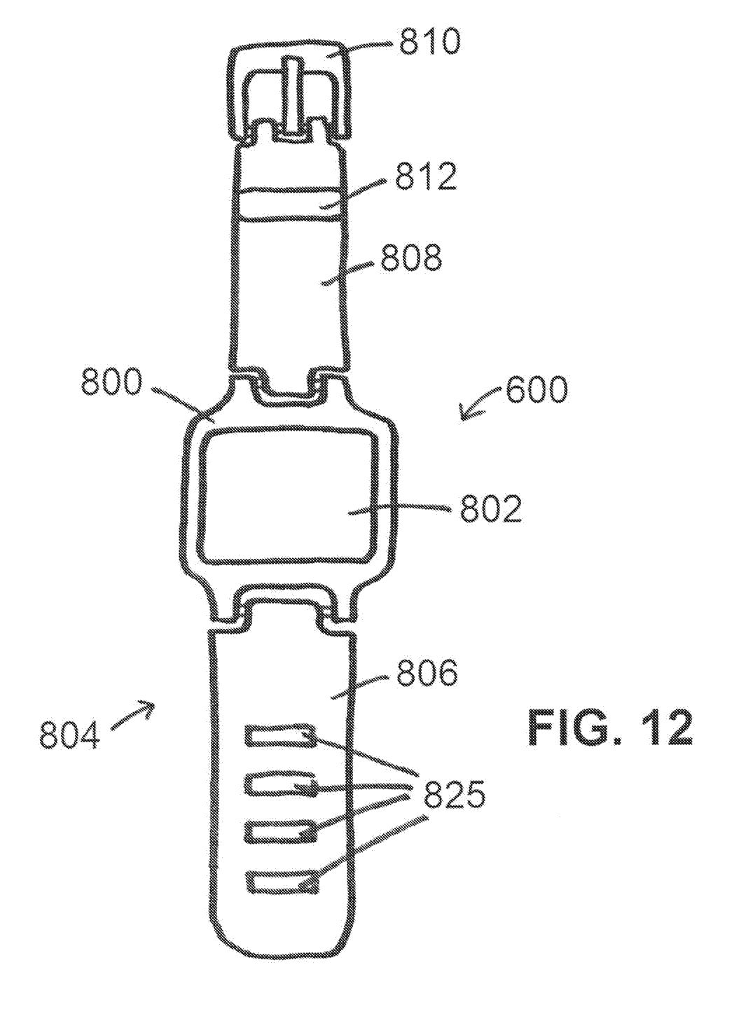

FIG. 12 shows an exemplary system 300 of the present disclosure. An exemplary system 300 of the present disclosure may comprise a first device 600. Exemplary first devices 600 of the present disclosure may comprise a smartwatch, namely a wrist-worn device configured to run various applications (or “apps,” namely exemplary software 116 of the present disclosure) configured to run on exemplary system hardware 210, such as a client 112 configured as a smartwatch.

Exemplary first devices 600 of the present disclosure are also configured to tighten around a wrist of a user/wearer to measure blood pressure from the wrist at preschedule times (e.g., morning and evening). Exemplary first devices 600 (such as a smartwatches), such as shown in a top view shown in FIG. 12, comprise a watch housing 800 and a display 802 (which can be, for example, a display screen 224) mounted within or otherwise coupled to watch housing 800. Various “computer” components, such as components of systems 300 or clients 112 referenced herein, such as a central processor 214, various storage media referenced herein, etc., can be contained within watch housing 800. Watch housing 800 is also coupled to, or otherwise positioned adjacent to, a watch band 804, which can comprise a first band portion 806 and/or a second band portion 808. Exemplary first devices 600 of the present disclosure can also comprise additional traditional watch elements, such as, for example, a clasp portion 810 coupled to second band portion 808, whereby clasp portion 810 is configured to engage at least part of first band portion 806 so that first device 600 can be worn on a wrist, and a retainer 812 coupled to or otherwise positioned adjacent to second band portion 808, whereby retainer 812 is configured to receive at least part of first band portion 806 to hold part of first band portion 806 in place next to portions of second band portion 808.

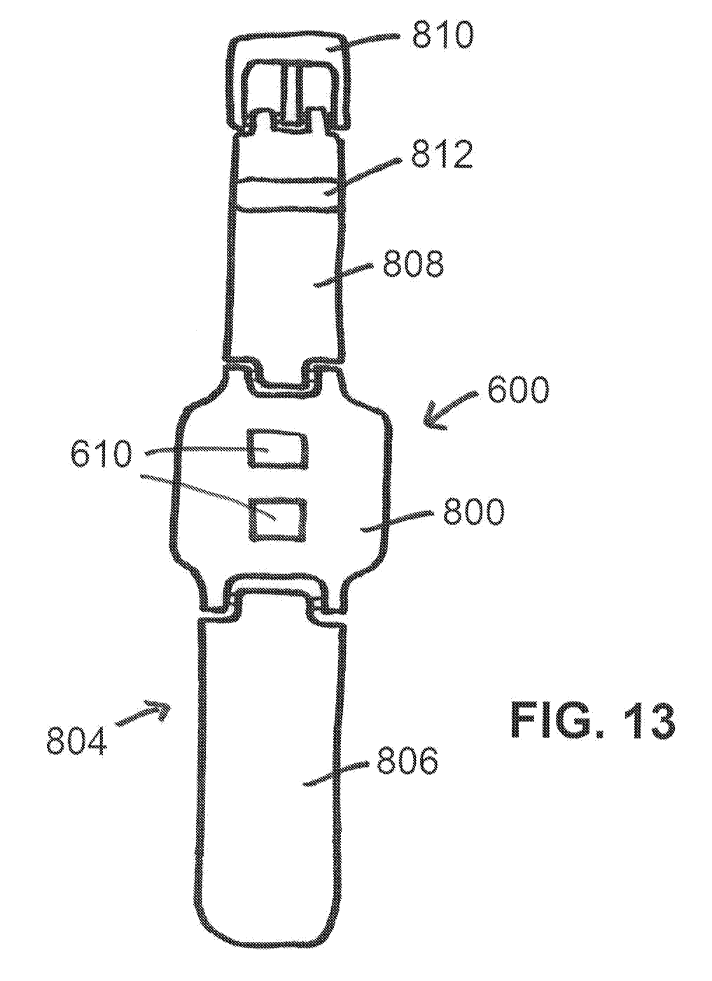

Exemplary first devices 600 (such as a smartwatches), such as shown in the back/underside view shown in FIG. 13, may comprise one or more sensors or electrodes 610, noting that one, two, three, or more sensors or electrodes 610 could comprise part of first device 600. Said sensors or electrodes 610, as referenced herein, can be configured as blood pressure sensors or electrodes, whereby said sensors or electrodes 610 can obtain blood pressure data from blood vessel(s) within a wrist of a wearer of said first device 600.

Tightening of first device 600 can be calibrated to the user so that excess constriction can be avoided during operation of said first devices 600 when obtaining blood pressure measurements.

Blood pressure measurements can be made from the radial artery in the wrist if a watch (an exemplary first device 600) is made to constrict similar to a traditional blood pressure cuff used conventionally at the brachial artery. The wrist watch band 804 can be made to be pulled (manually or automatically) in at controlled notches 825 (increments), such as shown in FIG. 12, that are calibrated for mmHg pressure changes; i.e., notch constriction of wrist watch (first device 600) to cuff at the bicep in terms of mmHg. This patient-specific calibration of wrist constriction to mmHg cuff inflation can be used to overcome the need for a mercury manometer. Once this is established, the principle of wrist blood pressure measurement is the same as that of brachial pressure where the constriction is increased to the point of full radial artery occlusion followed by gradual release to detect (with vibration sensor) first opening (systolic value) followed by full opening (diastolic value).

As noted above, tightening can be manual or automatic. In embodiments where the tightening is manual, for example, a wearer of the smartwatch (first device 600) can wear the first device 600 at a first setting, and can manually tighten first device 600 about/around the wrist to a second setting, whereby the second setting is tighter than the first setting, namely whereby first device 600 is more tightly positioned about/around the wrist at the second setting than the first setting. The first and second settings can be adjusted by way of engaging different notches 825 of first band portion 806 by clasp portion 810, for example. When first device 600 is worn relatively tight about/around the wrist, sensors or electrodes 610 of first device 600 can be operated to obtain blood pressure measurements.

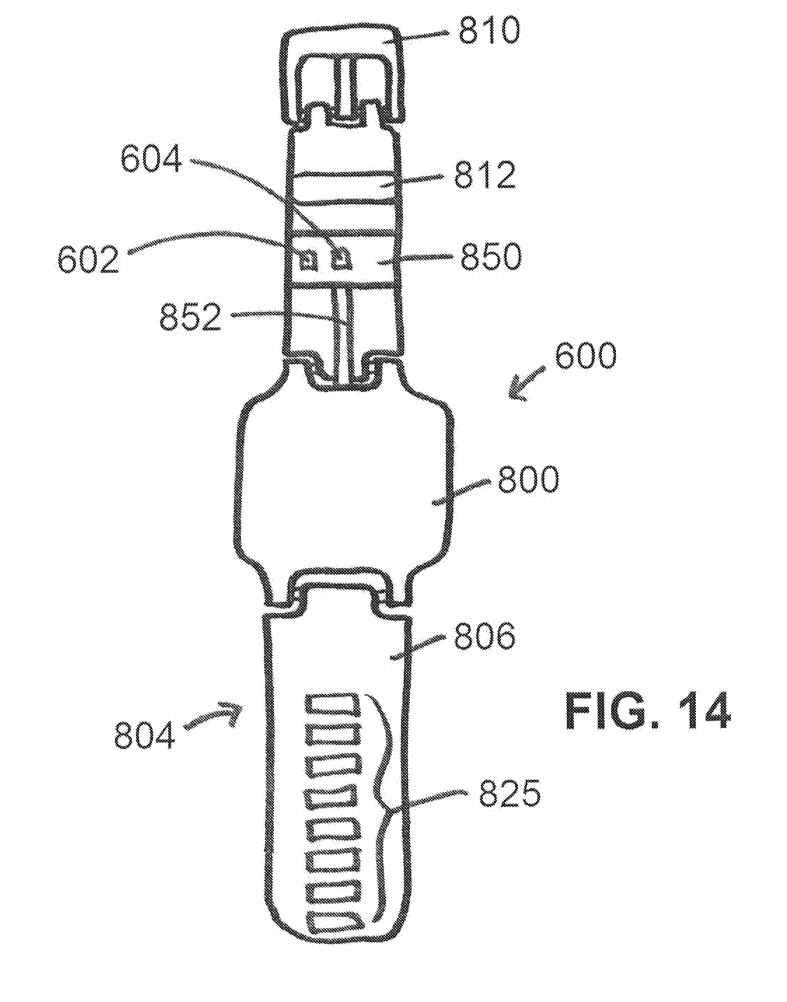

Various first device 600 embodiments of the present disclosure, configured as smartwatches, can also be tightened automatically. For example, and as shown in FIG. 14, an exemplary first device 600 of the present disclosure comprises a gear mechanism 850 coupled to second band portion 808, whereby gear mechanism 850 is configured to receive at least part of first band portion 806 and engage one or more notches 825 of first band portion 806 so to tighten or loosen watch band 804 of first device 600 as may be desired. For example, should first device 600 be set to obtain a blood pressure measurement, gear mechanism 850, which would be in communication with central processor 214 of first device 600, either wireles sly or wired (such as by wire 852), could be directed to operate by central processor 214 so to tighten watch band 804 about/around the wearer's wrist (such as to a second setting) so that sensors or electrodes 610 could be operated to obtain blood pressure data. After the blood pressure data is obtained, central processor 214 could direct gear mechanism 850 to operate to loosen watch band 804 (such as back to a first setting) so that watch band 804 is no longer tight (at the second setting). Gear mechanisms 850 of the present disclosure could have various components such as a first device 600, such as a client 112 of the present disclosure, including, but not limited to, a wired or wireless receiver 602 and/or wireless transmitter 604.

Frequent recordings of blood pressure can improve management of hypertension (more frequent titrations of medication) for improved health. Measurements of pressure several times per day, for example, can populate a database (referenced in further detail below) to monitor risk factors and patient health for prevention and early intervention.

As referenced above, a first device 600 can obtain blood pressure data/information from the body, by way of operation of sensors or electrodes 610 configured to obtain said blood pressure data/information. Said blood pressure data/information, obtained by first device 600 (such a smartwatch), could be recorded by an app (software 116) on said first device 600. Said data could then be processed by system 300, as follows.

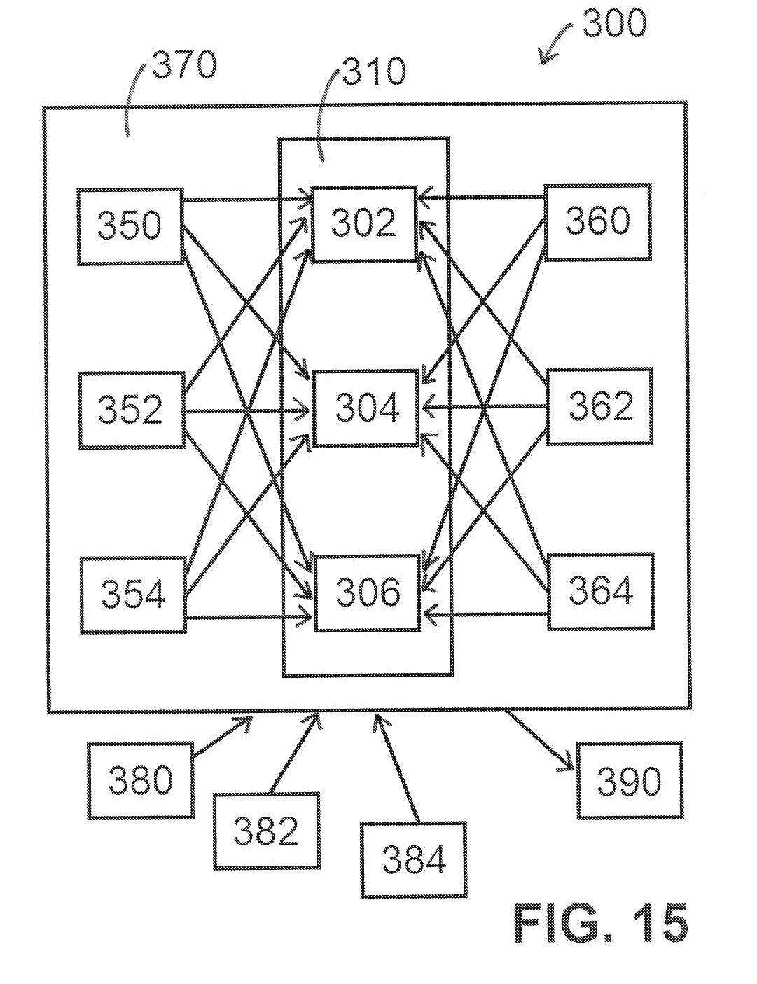

In at least one system 300 embodiment, such as shown in FIG. 15, system 300 uses a plurality of computational simulations, such as a first computational simulation 302, a second computational simulation 304, and a third computational simulation 306. Additional computation simulations could also be used by system 300, as described in further detail herein. Said computational simulations, such as computational simulations 302, 304, 306, etc., can comprise computational simulations within a library 310. Library 310, therefore, would include at least two computational simulations, and in at least some embodiments more than two, such as simulations 302, 304, 306, etc. Computational simulations 302, 304, 306, etc., as referenced herein, may include any number of simulations, including those relating to blood flow, vessel geometry, simulations of blood flow, and the like.

The aforementioned computational simulations 302, 304, 306, etc., are generated using generic information and patient information. For example, any number of patient simulations 302, 304, 306, etc., could be generated using a combination of any of first generic information 350, second generic information 352, third generic information 354, additional generic information, and any of first model patient information 360, second model patient information 362, third model patient information 364, and/or additional model patient information. As shown in FIG. 15, for example, said computational simulations 302, 304, 306 comprising any number of generic information 350, 352, 354 and any number of model patient information 360, 362, 364, can collectively form combined simulation 370.

Generic information 350, 352, 354, etc., can comprise, for example, field equations (laws of mechanics, conservation of mass, momentum, and/or energy), such as those using a geometric mesh on which the computations are performed, for example, including but not limited to information that is generic to the model/simulation. Model patient information 360, 362, 364, etc., can comprise, for example patient-specific geometry or structure of organ of interest. For example, and for a blood vessel, it could be the geometry of the vessel, and for the heart, it could be the geometry of left or right ventricles, etc. Said model patient information 360, 362, 364, etc., can also involve material properties or mechanical properties of the patient-specific system (i.e., stress-strain relation), and/or use boundary or loading conditions; e.g., patient blood pressure in the vessel or ventricle.

Combined simulation 370, as shown in FIG. 15, or one or more individual patient simulations 302, 304, 306, etc., can be used as follows. A patient of interest, as referenced herein, can be evaluated, tested, analyzed, etc., so that one or more patient-specific parameters can be obtained, such as a first patient-specific parameter 380, a second patient-specific parameter 382, a third patient-specific parameter 384, etc., which are therefore specific to the patient of interest, such as, for example, any number of the anatomy, material properties, and/or boundary conditions referenced herein. The blood pressure data/information obtained using first device 600, for example, could be one or more of these parameters 380, 382, 384, etc., and processed according to the present disclosure, as may be desired. One or more of the patient-specific parameters 380, 382, 384 can be analyzed in view of any of computation simulations 302, 304, 306, etc., or combined simulation 370, such as shown in FIG. 15, so to generate patient-specific outcome 390.

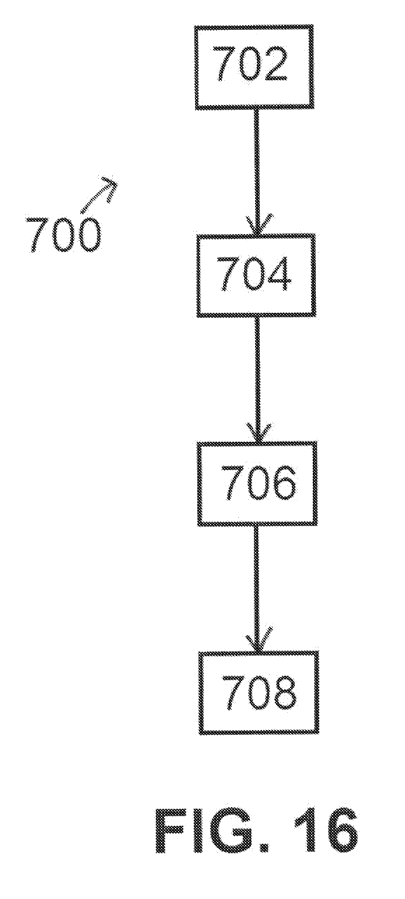

An exemplary method 700 of the present disclosure is shown in block step format in FIG. 16. As shown in FIG. 16, an exemplary method 700 of the present disclosure includes the step of operating a smartwatch (an exemplary first device 600) having an app (software 116) stored thereon or accessibly therefrom, whereby the smartwatch (first device 600) has one or more sensors or electrodes 610 that are operated to obtain blood pressure data/information from the user of first device 600 (an exemplary operation step 702). Method 700 may further comprise the step of comparing the blood pressure data/information with other information within a library 310 or to prior blood pressure data/information (an exemplary data processing step 704), and should any (or a subset of) the blood pressure data/information indicate a blood pressure measurement below, near, at, or above a threshold measurement level, alerting the user of the first device 600, whereby the alert is provided by first device 600 itself (an exemplary alerting step 706). For example, blood pressure measurements at or below a threshold level could be an indicator of potential hypotension or other disease indicative of low blood pressure, and blood pressure measurements at or above a threshold level could be an indicator of potential hypertension or cardiovascular disease. Should the user be alerted by step 706, such as by some sort of visual and/or auditory signal provided by first device 600, the user could then seek medical attention and potential treatment (an exemplary medical attention step 708).

First devices 600 of the present disclosure, configured as a smartwatch, can perform additional tasks other than obtaining blood pressure measurements, such as displaying the date and/or time, displaying other “apps” (software 116), and performing various functions of other smartwatches available in the marketplace.

An assessment of the severity of a potentially high blood pressure reading can provide early detection of disease relating to high blood pressure, such as hypertension and cardiovascular disease. Alternatively, low blood pressure readings could provide early detection of disease relating to the same, such as low blood pressure.

In view of the foregoing, the present disclosure includes disclosure of devices and systems configured to obtain and assess blood pressure measurements, alert the user of said devices or systems of the same, to that the user can seek medical attention if necessary/desired to address the high or low blood pressure situation.

While various embodiments of real-time therapy-aided simulation libraries and methods to construct and use the same, embodiments of continuous individual-specific risk factor assessments and methods to construct and use the same, embodiments of assessments of a carotid stenosis using a smartphone and methods to use the same, and embodiments of assessments of blood pressure using a smartwatch and methods to use the same have been described in considerable detail herein, the embodiments are merely offered as non-limiting examples of the disclosure described herein. It will therefore be understood that various changes and modifications may be made, and equivalents may be substituted for elements thereof, without departing from the scope of the present disclosure. The present disclosure is not intended to be exhaustive or limiting with respect to the content thereof.

Further, in describing representative embodiments, the present disclosure may have presented a method and/or a process as a particular sequence of steps. However, to the extent that the method or process does not rely on the particular order of steps set forth therein, the method or process should not be limited to the particular sequence of steps described, as other sequences of steps may be possible. Therefore, the particular order of the steps disclosed herein should not be construed as limitations of the present disclosure. In addition, disclosure directed to a method and/or process should not be limited to the performance of their steps in the order written. Such sequences may be varied and still remain within the scope of the present disclosure.

1. A method, performed using a processor in communication with a first storage device to execute instructions stored thereon, the method comprising the steps of:
accessing a combined simulation stored on the first storage device or a second storage device, the combined simulation comprising a library comprising a plurality of computational simulations;
obtaining a first patient-specific parameter and a second patient-specific parameter from a patient; and
analyzing the first patient-specific parameter and the second patient-specific parameter in view of the combined simulation to generate a patient-specific outcome comprising a first outcome element and a second outcome element.

2. The method of claim 1, further comprising the step of:
performing a medical procedure upon the patient based upon the patient-specific outcome.

3. The method of claim 1, wherein the plurality of computational simulations is generated using generic information and model patient information.

4. The method of claim 1, wherein the generic information comprises one or more of field equations, and wherein the model patient information comprises a patient-specific geometry or structure of an organ or a vessel.

5. The method of claim 1, wherein at least one computational simulation of the plurality of computational simulation is selected from the group consisting of a cardiovascular simulation, a cardiac simulation, a blood flow simulation, a stent simulation, a stent graft simulation, and a cardiac mechanics simulation.

6. The method of claim 1, wherein a computational simulation of the plurality of computational simulations comprises a first aspect relating to patient age and a second aspect relating to patient weight.

7. A method, performed using a processor in communication with a first storage device to execute instructions stored thereon, the method comprising the steps of:
accessing an electronic medical record on the first storage device or a second storage device, the electronic medical record comprising a first patient-specific parameter, a second patient-specific parameter, and a third patient-specific parameter;
generating a risk factor baseline based upon the electronic medical record and reporting a risk factor should a risk factor be identified upon generation of the risk factor baseline;
operating a portable device to receive patient data from a remote device comprising one or more sensors or electrodes positioned upon or within a patient;
incorporating the patient data into the electronic medical record to form an updated electronic medical record; and
generating a risk factor level based upon the updated electronic medical record and reporting a risk factor change or a new risk factor should the risk factor change or the new risk factor be identified upon generation of the risk factor level.

8. The method of claim 7, wherein the first patient-specific parameter is selected from the group consisting of a heart rate, a blood pressure, a blood glucose level, and a cholesterol level.

9. The method of claim 7, wherein the first patient-specific parameter is historical or current.

10. The method claim 7, wherein the portable device is selected from the group consisting of a smartphone and a smartwatch.

11. The method of claim 7, further comprising the steps of:
operating the portable device again to receive additional patient data from the remote device;
incorporating the additional patient data into the electronic medical record to form a further updated electronic medical record; and
generating an additional risk factor level based upon the further updated electronic medical record and reporting an additional risk factor change or an additional new risk factor should the additional risk factor change or the additional new risk factor be identified upon generation of the additional risk factor level.

12. A method, performed using a processor in communication with a first storage device to execute instructions stored thereon, the method comprising the steps of:
operating a first device comprising one or more sensors or electrodes configured to detect physiological information so to obtain physiological information/data from a user;
comparing the physiological information/data with library data or with previously-obtained data to generate a comparison result; and
alerting the user should the comparison result be at or above a first threshold level.

13. The method of claim 12, wherein the physiological information comprises sound vibrations and/or turbulence created by stenosis or constriction of a vessel lumen, wherein the physiological information/data comprises vibration and/or turbulence data, and wherein the comparison result identifies a carotid stenosis or a degree of carotid stenosis near, at, or above the first threshold level.

14. The method of claim 13, further comprising the step of:
obtaining medical attention and treatment based upon the identified carotid stenosis or the degree of carotid stenosis near, at, or above the first threshold level.

15. The method of claim 13, wherein the step of comparing is performed to compare the vibration and/or turbulence data with library data comprising a plurality of computational simulations generated using generic information and model patient information.

16. The method of claim 12, wherein the physiological information comprises blood pressure, wherein the physiological information/data comprises blood pressure data/information, and wherein the comparison result identifies a blood pressure measurement near, at, or above the first threshold level or at, near, or below a second, lower threshold level.

17. The method of claim 16, further comprising the step of:
obtaining medical attention and treatment for hypotension should the comparison result identify a blood pressure measurement at, near, or below the second, lower threshold level.

18. The method of claim 16, further comprising the step of:
obtaining medical attention and treatment for hypertension or cardiovascular disease should the comparison result identify a blood pressure measurement at, near, or above the first threshold level.

19. The method of claim 16, wherein the step of comparing is performed to compare the blood pressure data/information with library data comprising a plurality of computational simulations generated using generic information and model patient information.

20. The method of claim 16, wherein the first device is configured as a smartwatch, and wherein the step of operating the first device is performed while the first device is worn upon a wrist of the user.
